(19) United States
(12) Patent Application Publication
(10) Pub. No.: US 2020/0289027 A1
(43) Pub. Date: Sep. 17, 2020

(54) SYSTEM, METHOD AND COMPUTER PROGRAM PRODUCT FOR ASSESSMENT OF A USER'S GAIT

(71) Applicant: CELLOSCOPE LTD., TEL AVIV (IL)

(72) Inventors: Yuval NAVEH, Haifa (IL); Shahar DAVIDSON, Rehovot (IL); Tomer SHUSSMAN, Kfar Saba (IL)

(21) Appl. No.: 16/659,832

(22) Filed: Oct. 22, 2019

Related U.S. Application Data
(60) Provisional application No. 62/816,227, filed on Mar. 11, 2019.

(51) Int. Cl. A61B 5/11; A61B 5/00; G06F 3/01

(52) U.S. Cl. CPC A61B 5/112; A61B 5/7264; A61B 2562/0219; G06F 3/011; G06F 3/017; A61B 5/0022

(57) ABSTRACT

A gait monitoring system operative to monitor gait of an end-user bearing a wearable device equipped with at least one magneto-inertial sensor, the system comprising a processor configured to receive raw sensor data from the wearable device's at least one magneto-inertial sensor to extract situational data from the raw sensor data, the situational data including at least the device's bodily position relative to the end-user, to determine a gait analysis process which yields at least one parameter characterizing the end-user's gait, depending at least on the device's bodily position as extracted, and to compute, and generate an output indication of, the at least one parameter characterizing the end-user's gait, by running the gait analysis process as selected.

### FIELD OF THIS DISCLOSURE

The present disclosure relates generally to computerized systems and more particularly to gait monitoring systems.

### BACKGROUND FOR THIS DISCLOSURE

Conventional technology constituting background to certain embodiments of the present invention is described in the following publications inter alia:

Gait analysis is known. Inter alia, the concept of partitioning gait into phases, is known. One commonly used partition includes the following phases or periods: 1) initial contact 2) loading response 3) mid stance 4) terminal stance 5) pre-swing 6) initial swing 7) mid swing 8) terminal swing.

Another option is to define only 2 phases: stance (typically including the first 4 above as “periods” within the stance phase) and swing (typically including the latter 4 above as “periods” within the swing phase).

Either of the above are merely example partitions of the gait cycle.

Prior art on gait analysis includes:
Jacquelin Perry, “Gait analysis: normal and pathological function.” (1992)
https://gaitup.com/
https://www.xsens.com/
Giansanti, Daniele, Giovanni Maccioni, and Velio Macellari. “The development and test of a device for the reconstruction of 3-D position and orientation by means of a kinematic sensor assembly with rate gyroscopes and accelerometers.” IEEE transactions on biomedical engineering 52.7 (2005): 1271-1277.
Gastaldi, Laura, et al. “Technical challenges using magneto-inertial sensors for gait analysis.” 2016 IEEE International Symposium on Medical Measurements and Applications (MeMeA). IEEE, 2016.
Teufl, Wolfgang, et al. “Towards inertial sensor based mobile gait analysis: event-detection and spatio-temporal parameters.” Sensors 19.1 (2019): 38.
Ellis, Robert J., et al. “A validated smartphone-based assessment of gait and gait variability in Parkinson's disease.” PLoS one 10.10 (2015): e0141694.
Cereatti, Andrea, Diana Trojaniello, and Ugo Della Croce. “Accurately measuring human movement using magneto-inertial sensors: techniques and challenges.” 2015 IEEE International Symposium on Inertial Sensors and Systems (ISISS) Proceedings. IEEE, 2015.
Wang, Jindong, et al. “Deep learning for sensor-based activity recognition: A survey.” Pattern Recognition Letters 119 (2019): 3-11.
Ordóñez, Francisco, and Daniel Roggen. “Deep convolutional and LSTM recurrent neural networks for multimodal. wearable activity recognition.” Sensors 16.1 (2016): 115.
Murad, Abdulmajid, and Jae-Young Pyun. “Deep recurrent neural networks for human activity recognition.” Sensors 17.11 (2017): 2556.
Gadaleta, Matteo, and Michele Rossi. “Idnet: Smartphone-based gait recognition with convolutional neural networks.” Pattern Recognition 74 (2018): 25-37.
Silsupadol, Patima, Kunlanan Teja, and Vipul Lugade. “Reliability and validity of a smartphone-based assessment of gait parameters across walking speed and smartphone locations: Body, bag, belt, hand, and pocket.” Gait & posture 58 (2017): 516-522.
(https://matlabgeeks.comAw-content/uploads12018/08/Silsupadol-2017-GP-Reliability-and-validity-of-a-smartphone-based-assessment-of-gait-parameters-across-walking-speed-and-smartphone-locations.pdf)
https://gaitup.com/
How, Tuck-Voon, et al. “MyWalk: a mobile app for gait asymmetry rehabilitation in the community.” Proceedings of the 7th International Conference on Pervasive Computing Technologies for Healthcare. ICST (Institute for Computer Sciences, Social-Informatics and Telecommunications Engineering), 2013.
U.S. Pat. No. 10,307,086 describes use of mobile devices for gait measurement.

Other known patent documents in the field of this disclosure include U.S. Pat. Nos. 9,700,241B2, 10,307,086, 10,231,651.

The concept of partitioning gait into phases is known. One commonly used partition includes the following phases or periods: 1) initial contact 2) loading response 3) mid stance 4) terminal stance 5) pre-swing 6) initial swing 7) mid swing 8) terminal swing.

Another option is to define only 2 phases: stance (typically including the first 4 above as “periods” within the stance phase) and swing (typically including the latter 4 above as “periods” within the swing phase).

Either of the above are merely example partitions of the gait cycle.

The disclosures of all publications and patent documents mentioned in the specification, and of the publications and patent documents cited therein directly or indirectly, are hereby incorporated by reference other than subject matter disclaimers or disavowals. If the incorporated material is inconsistent with the express disclosure herein, the interpretation is that the express disclosure herein describes certain embodiments, whereas the incorporated material describes other embodiments. Definition/s within the incorporated material may be regarded as one possible definition for the terms in question.

### SUMMARY OF CERTAIN EMBODIMENTS

Certain embodiments of the present invention seek to provide a system, typically seamless, typically with monitoring functionality, typically with assessment of gait quality, typically having capability for assessing correctness of a user's gait.

Certain embodiments of the present invention seek to provide circuitry typically comprising at least one processor in communication with at least one memory, with instructions stored in such memory executed by the processor to provide functionalities which are described herein in detail. Any functionality described herein may be firmware-implemented or processor-implemented, as appropriate.

It is appreciated that any reference herein to, or recitation of, an operation being performed is, e.g. if the operation is performed at least partly in software, intended to include both an embodiment where the operation is performed in its entirety by a server A, and also to include any type of “outsourcing” or “cloud” embodiments in which the operation, or portions thereof, is or are performed by a remote processor P (or several such), which may be deployed off-shore or “on a cloud”, and an output of the operation is then communicated to, e.g. over a suitable computer network, and used by, server A. Analogously, the remote processor P may not, itself, perform the entire operation, and, instead, the remote processor P itself may receive output/s of portion/s of the operation from yet another processor/s may be deployed off-shore relative to P, or “on a cloud”, and so forth.

There is thus provided, in accordance with at least one embodiment of the present invention, The present invention typically includes at least the following embodiments:

Embodiment 1. A system e.g. gait monitoring system operative to monitor gait of an end-user bearing a wearable device which may be equipped with at least one sensor e.g. magneto-inertial sensor, the system comprising all or any subset of:

a processor typically configured to (perform all or any subset of): receive data e.g. raw sensor data which may be provided from the wearable device's at least one magneto-inertial sensor, to extract situational data from the raw sensor data, the situational data typically including at least the device's bodily position relative to the end-user, to determine a gait analysis process which typically yields at least one parameter characterizing the end-user's gait, typically depending at least on the device's bodily position as extracted, and to compute, and generate an output indication of, the at least one parameter characterizing the end-user's gait, e.g. by running the gait analysis process as selected.

The wearable device typically includes a processing unit and at least one sensor e.g. magneto-inertial sensor.

The gait analysis process for different device bodily positions may differ e.g. in that for one bodily position, a certain gait parameter (is deemed available e.g. a system table indicates this parameter to be available for this bodily position and) is computed, whereas for another bodily position, the same gait parameter is not deemed (e.g. by system tables) available for this bodily position and therefore is not computed.

And/Or, different parameters and/or thresholds and/or algorithms and/or heuristics and/or assumptions and/or baselines and/or normal ranges may be employed for different bodily positions. For example, certain gait disorders may be available (discoverable) for a first device bodily position but not for a second device bodily position.

Embodiment 2. The system according to any of the preceding embodiments wherein the situational data also comprises a classification of a physical activity in which the end-user is engaging while the magneto-inertial sensor is recording the raw sensor data.

Embodiment 3. The system according to any of the preceding embodiments wherein, to extract the situational data, the processor operates a classifier which receives inputs including a stream of motion data or device acceleration data which may be derived from the magneto-inertial sensor and outputs one of plural classes for each input, and wherein at least some of the classes include an (end-user physical activity, device's bodily position) pair.

The motion data may for example include acceleration data which may be derived from the magneto-inertial sensor. The motion data typically comprises an online time sequence of at least 3 (typically) channels of rotation and/or 3 (typically) channels of acceleration (typically rotational acceleration) respectively corresponding to the x, y and z axes of the 3d world. Typically the x axis represents the axis along which the end-user and her/his device is moving, the y axis represents up-down (for example: swing has a vertical (y) component) and the z axis represents right-left. Typically, due to standardization or normalization e.g. as described herein, the z axis indeed represents right-left relative to the user's direction of motion, rather than representing east-west as may he the case for z-axis components of the raw data.

Embodiment 4. The system according to any of the preceding embodiments wherein the wearable device comprises a networked communication device.

Network communication is useful for some applications such as communicating in real time with emergency services and occasional updates of the software (e.g. to synchronize with updates in the API via which the system herein communicates with the wearable device's sensors), however, it is not needed for alerting\notifying\giving feedbacks to end-user.

Embodiment 5. The system according to any of the preceding embodiments wherein the processor includes logic which is responsive to each receipt of the raw sensor data and wherein the processor is operative to extract, select, and compute, triggered by the logic.

Embodiment 6. The system according to any of the preceding embodiments wherein the logic, in at least some operational modes, triggers the processor to extract, select and compute, responsive to less than all instances of receipt of the raw sensor data.

Embodiment 7. The system according to any of the preceding embodiments wherein at least one operational mode is provided whose internal logic triggers the processor to extract, selects and computes responsive to each instance of receipt of the raw sensor data.

Embodiment 8. The system according to any of the preceding embodiments wherein the parameter characterizing the end-user's gait comprises an indication of whether the end-user's gait is characteristic of an end-user who is undergoing a stroke.

Embodiment 9. The system according to any of the preceding embodiments the parameter characterizing the end-user's gait comprises the end-user's walking pace.

Embodiment 10. The system according to any of the preceding embodiments wherein the parameter characterizing the end-user's gait comprises the end-user's asymmetry.

Embodiment 11. The system according to any of the preceding embodiments wherein the parameter characterizing the end-user's gait comprises the end-user's cadence.

Embodiment 12. The system according to any of the preceding embodiments wherein the parameter characterizing the end-user's gait comprises the end-user's stride length.

Embodiment 13. The system according to any of the preceding embodiments wherein the gait analysis process selected for a first bodily position extracts at least one parameter characterizing the end-user's gait, which is not extracted by the gait analysis process selected for a second bodily position.

for example, the end user's thigh's maximal rotation may be available for extraction when the device is in the end-user's front pocket, but not when the device is in the end-user's hand.

Embodiment 14. The system according to any of the preceding embodiments wherein the system stores an activity-specific baseline value for at least one parameter P characterizing the end-user's gait and wherein the output indication comprises an indication of whether an end-user's gait's current value for P has strayed from the activity-specific baseline value stored specifically for the end-user and specifically for the activity in which the end-user is currently engaged.

for example, an end-user's stride cadence may differ depending on whether s/he is climbing stairs or walking.

Embodiment 15. The system according to any of the preceding embodiments wherein the system stores a device's bodily position-specific baseline value for at least one parameter P characterizing the end-user's gait and wherein the output indication comprises an indication of whether an end-user's gait's current value for P has strayed from the device's bodily position-specific baseline value stored specifically for the end-user and specifically for the current device's bodily position as extracted.

Embodiment 16. The system according to any of the preceding embodiments wherein the system stores an (end-user physical activity, device's bodily position) pair-specific baseline value for at least one parameter P characterizing the end-user's gait and wherein the output indication comprises an indication of whether an end-user's gait's current value for P has strayed from the baseline value stored specifically for the end-user and specifically for the (end-user physical activity, device's bodily position) pair most recently output by the classifier.

Embodiment 17. The system according to any of the preceding embodiments wherein the wearable device comprises a cellular phone.

Embodiment 18. The system according to any of the preceding embodiments and wherein the output indication includes at least one graph, in at least one spatial dimension, of the end-user's average stride as though the end-user were striding in place or on a treadmill.

The system may derive e.g. from the graph at least one discontinuous point along the graph e.g. at least one point (e.g. the point of foot contact within the cycle characterized by low speed and/or change of direction)) at which the graph's derivative changes suddenly or discontinuously rather than continuously and may use this discontinuous point in analysis e.g. as a uniform starting point used for all graphs (e.g. when partitioning the graphs into 100) or when computing secondary parameters.

Embodiment 19. The system according to any of the preceding embodiments wherein the graph comprises a closed curve.

Embodiment 20. The system according to any of the preceding embodiments wherein the at least one graph in at least one spatial dimension comprises 3 two-dimensional graphs.

Embodiment 21. The system according to any of the preceding embodiments wherein the processor includes internal logic which is responsive to each receipt of the raw sensor data and wherein the processor is operative to extract, select and compute, triggered by the internal logic.

The internal logic may, at least in some operational modes, trigger the processor to extract, select and compute responsive to less than all instances of receipt of the raw sensor data. At least one operational mode may be provided in which the internal logic triggers the processor to extract, select and compute responsive to each instance of receipt of the raw sensor data.

Embodiment 22: A gait analysis system including:

a processor which computationally manipulates data e.g. raw sensor data typically describing an end-user's gait, to obtain at least one graph, typically in at least one spatial dimension, of a stride e.g. the end-user's average stride as though the end-user were striding in place or on a treadmill; and/or

an output device which generates an output indication of the at least one graph.

Embodiment 23. A gait monitoring method operative to monitor gait of an end-user bearing a wearable device equipped with at least one magneto-inertial sensor, the method comprising providing a processor configured to receive raw sensor data from the wearable device's at least one magneto-inertial sensor to extract situational data from the raw sensor data, the situational data including at least the device's bodily position relative to the end-user, to determine gait analysis functionality which yields at least one parameter characterizing the end-user's gait, depending at least on the device's bodily position as extracted, and to compute, and generate an output indication of, the at least one parameter characterizing the end-user's gait, by running the gait analysis process as selected.

Embodiment 24: A computer program product, comprising a non-transitory tangible computer readable medium having computer readable program code embodied therein, the computer readable program code adapted to be executed to implement any method shown and described herein.

Also provided, excluding signals, is a computer program comprising computer program code means for performing any of the methods shown and described herein when the program is run on at least one computer; and a computer program product, comprising a typically non-transitory computer-usable or -readable medium e.g. non-transitory computer-usable or -readable storage medium, typically tangible, having a. computer readable program code embodied therein, the computer readable program code adapted to he executed to implement any or all of the methods shown and described herein. The operations in accordance with the teachings herein may be performed by at least one computer specially constructed for the desired purposes or general purpose computer specially configured for the desired purpose by at least one computer program stored in a typically non-transitory computer readable storage medium. The term “non-transitory” is used herein to exclude transitory, propagating signals or waves, but to otherwise include any volatile or non-volatile computer memory technology suitable to the application.

Any suitable processor/s, display and input means may be used to process, display e.g. on a computer screen or other computer output device, store, and accept information such as information used by or generated by any of the methods and apparatus shown and described herein; the above processor/s, display and input means including computer programs, in accordance with all or any subset of the embodiments of the present invention. Any or all functionalities of the invention shown and described herein, such as but not limited to operations within flowcharts, may be performed by any one or more of: at least one conventional personal computer processor, workstation or other programmable device or computer or electronic computing device or processor, either general-purpose or specifically constructed, used for processing; a computer display screen and/or printer and/or speaker for displaying; machine-readable memory such as flash drives, optical disks, CDROMs, DVDs, BluRays, magnetic-optical discs or other discs; RAMs, ROMs, EPROMs, EEPROMs, magnetic or optical or other cards, for storing, and keyboard or mouse for accepting. Modules illustrated and described herein may include any one or combination or plurality of: a server, a data processor, a memory/computer storage, a communication interface (wireless (e.g. BLE) or wired (e.g. USB)), a computer program stored in memory/computer storage.

The term “process” as used above is intended to include any type of computation or manipulation or transformation of data represented as physical, e.g. electronic, phenomena which may occur or reside e.g. within registers and/or memories of at least one computer or processor. Use of nouns in singular form is not intended to be limiting; thus the term processor is intended to include a plurality of processing units which may be distributed or remote, the term server is intended to include plural typically interconnected modules running on plural respective servers, and so forth.

The above devices may communicate via any conventional wired or wireless digital communication means, e.g. via a wired or cellular telephone network or a computer network such as the Internet.

The apparatus of the present invention may include, according to certain embodiments of the invention, machine readable memory containing or otherwise storing a program of instructions which, when executed by the machine, implements all or any subset of the apparatus, methods, features and functionalities of the invention shown and described herein. Alternatively or in addition, the apparatus of the present invention may include, according to certain embodiments of the invention, a program as above which may be written in any conventional programming language, and optionally a machine for executing the program such as but not limited to a general purpose computer which may optionally be configured or activated in accordance with the teachings of the present invention. Any of the teachings incorporated herein may, wherever suitable, operate on signals representative of physical objects or substances.

The embodiments referred to above, and other embodiments, are described in detail in the next section.

Any trademark occurring in the text or drawings is the property of its owner and occurs herein merely to explain or illustrate one example of how an embodiment of the invention may be implemented.

Unless stated otherwise, terms such as, “processing”, “computing”, “estimating”, “selecting”, “ranking”, “grading”, “calculating”, “determining”, “generating”, “reassessing”, “classifying”, “generating”, “producing”, “stereo-matching”, “registering”, “detecting”, “associating”, “superimposing”, “obtaining”, “providing”, “accessing”, “setting” or the like, refer to the action and/or processes of at least one computer/s or computing system/s, or processor/s or similar electronic computing device/s or circuitry, that manipulate and/or transform data which may be represented as physical, such as electronic, quantities e.g. within the computing system's registers and/or memories, and/or may be provided on-the-fly, into other data which may be similarly represented as physical quantities within the computing system's memories, registers or other such information storage, transmission or display devices or may be provided to external factors e.g. via a suitable data network. The term “computer” should be broadly construed to cover any kind of electronic device with data processing capabilities, including, by way of non-limiting example, personal computers, servers, embedded cores, computing system, communication devices, processors (e.g. digital signal processor (DSP), microcontrollers, field programmable gate array (FPGA), application specific integrated circuit (ASIC), etc.) and other electronic computing devices. Any reference to a computer, controller or processor is intended to include one or more hardware devices e.g. chips, which may be co-located or remote from one another. Any controller or processor may for example comprise at least one CPU, DSP, FPGA or ASIC, suitably configured in accordance with the logic and functionalities described herein.

Any feature or logic or functionality described herein may be implemented by processor/s or controller/s configured as per the described feature or logic or functionality, even if the processor/s or controller/s are not specifically illustrated for simplicity. The controller or processor may he implemented in hardware, e.g., using one or more Application-Specific Integrated Circuits (ASICs) or Field-Programmable Gate Arrays (FPGAs) or may comprise a microprocessor that runs suitable software, or a combination of hardware and software elements.

The present invention may be described, merely for clarity, in terms of terminology specific to, or references to, particular programming languages, operating systems, browsers, system versions, individual products, protocols and the like. It will be appreciated that this terminology or such reference/s is intended to convey general principles of operation clearly and briefly, by way of example, and is not intended to limit the scope of the invention solely to a particular programming language, operating system, browser, system version, or individual product or protocol. Nonetheless, the disclosure of the standard or other professional literature defining the programming language, operating system, browser, system version, or individual product or protocol in question, is incorporated by reference herein in its entirety.

Elements separately listed herein need not be distinct components and alternatively may be the same structure. A statement that an element or feature may exist is intended to include (a) embodiments in which the element or feature exists; (b) embodiments in which the element or feature does not exist; and (c) embodiments in which the element or feature exist selectably e.g. a user may configure or select whether the element or feature does or does not exist.

Any suitable input device, such as but not limited to a sensor, may be used to generate or otherwise provide information received by the apparatus and methods shown and described herein. Any suitable output device or display may be used to display or output information generated by the apparatus and methods shown and described herein.

Any suitable processor/s may be employed to compute or generate or route, or otherwise manipulate or process information as described herein and/or to perform functionalities described herein and/or to implement any engine, interface or other system illustrated or described herein. Any suitable computerized data storage e.g. computer memory may be used to store information received by or generated by the systems shown and described herein. Functionalities shown and described herein may be divided between a server computer and a plurality of client computers. These or any other computerized components shown and described herein may communicate between themselves via a suitable computer network.

The system shown and described herein may include user interface/s e.g. as described herein which may for example include all or any subset of: an interactive voice response interlace, automated response tool, speech-to-text transcription system, automated digital or electronic interface having interactive visual components, web portal, visual interface loaded as web pager/s or screen/s from server/s via communication network/s to a web browser or other application downloaded onto a user's device, automated speech-to-text conversion tool, including a front-end interface portion thereof and back-end logic interacting therewith. Thus the term user interface or “UI” as used herein includes also the underlying logic which controls the data presented to the user e.g. by the system display and receives and processes and/or provides to other modules herein, data entered by a user e.g. using her or his workstation/device.

### BRIEF DESCRIPTION OF THE DRAWINGS

Example embodiments are illustrated in the various drawings. Specifically:

FIG. 1 illustrates logic blocks, all or any subset of which may be provided, each of which may be performed by suitable circuitry e.g. in software.

FIG. 2A illustrates raw data describing the device's motion, and FIGS. 2B (rotation) and 2c (acceleration) illustrate an example measurement result showing the six different measures of FIG. 2A.

FIGS. 3a-3b illustrate segmentation of the motion record of FIG. 2B, into strides.

The closed-cycle graphs of FIGS. 4a-5d each represent a given end-user's average “repetitive pattern”. FIG. 5a is a graph in accordance with an embodiment, of an end-user's stride when climbing stairs, with his mobile device in his left front pocket.

FIG. 5b is a graph in accordance with an embodiment, of an end-user's stride when going down stairs, with his mobile device in his right front pocket.

FIG. 5c is a graph in accordance with an embodiment, of an end-user's stride when walking, with his mobile device in his right back pocket.

FIG. 5d is a graph in accordance with an embodiment, of an end-user's stride when walking, with his mobile device held in his right hand.

FIGS. 6a and 6b each illustrate a reconstruction of a walking end-user's single stride atop aligned videos of the walking end-user.

FIG. 7a includes side, top and front view graphs of a reconstruction of circumduction gait. FIG. 7b includes side, top and front view graphs of a reconstruction of hiking gait.

FIGS. 8a-8b, taken together (aka “FIG. 8) form a simplified flowchart illustration of a gait analysis method provided in accordance with certain embodiments. The method of FIGS. 8a-8b typically comprises all or any subset of the illustrated operations, suitably ordered e.g. as shown.

Certain embodiments of the present invention are illustrated in the drawings; in the block diagrams, arrows between modules may be implemented as APIs and any suitable technology may be used for interconnecting functional components or modules illustrated herein in a suitable sequence or order e.g. via a suitable API/Interface. For example, state of the art tools may be employed, such as but not limited to Apache Thrift and Avro which provide remote call support. Or, a standard communication protocol may be employed, such as but not limited to HTTP or MQTT, and may be combined with a standard data format, such as but not limited to JSON or XML.

Methods and systems included in the scope of the present invention may include any subset or all of the functional blocks shown in the specifically illustrated implementations by way of example, in any suitable order e.g. as shown. Flows may include all or any subset of the illustrated operations, suitably ordered e.g. as shown. Tables herein may include all or any subset of the fields and/or records and/or cells and/or rows and/or columns described.

In the swim-lane diagrams, it is appreciated that any order of the operations shown may be employed rather than the order shown, however, preferably, the order is such as to allow utilization of results of certain operations by other operations by performing the former before the latter, as shown in the diagram.

Computational, functional or logical components described and illustrated herein can be implemented in various forms, for example, as hardware circuits such as but not limited to custom VLSI circuits or gate arrays or programmable hardware devices such as but not limited to FPGAs, or as software program code stored on at least one tangible or intangible computer readable medium and executable by at least one processor, or any suitable combination thereof A specific functional component may be formed by one particular sequence of software code, or by a plurality of such, which collectively act or behave or act as described herein with reference to the functional component in question. For example, the component may be distributed over several code sequences such as but not limited to objects, procedures, functions, routines and programs and may originate from several computer files which typically operate synergistically.

Each functionality or method herein may he implemented in software (E.g. for execution on suitable processing hardware such as a microprocessor or digital signal processor), firmware, hardware (using any conventional hardware technology such as Integrated Circuit technology) or any combination thereof.

Functionality or operations stipulated as being software-implemented may alternatively be wholly or fully implemented by an equivalent hardware or firmware module and vice-versa. Firmware implementing functionality described herein, if provided, may be held in any suitable memory device and a suitable processing unit (aka processor) may be configured for executing firmware code. Alternatively, certain embodiments described herein may be implemented partly or exclusively in hardware in which case all or any subset of the variables, parameters, and computations described herein may be in hardware.

Any module or functionality described herein may comprise a suitably configured hardware component or circuitry. Alternatively or in addition, modules or functionality described herein may be performed by a general purpose computer or more generally by a suitable microprocessor, configured in accordance with methods shown and described herein, or any suitable subset, in any suitable order, of the operations included in such methods, or in accordance with methods known in the art.

Any logical functionality described herein may be implemented as a real time application, if and as appropriate, and which may employ any suitable architectural option, such as but not limited to FPGA, ASIC or DSP or any suitable combination thereof.

Any hardware component mentioned herein may in fact include either one or more hardware devices e.g. chips, which may be co-located or remote from one another.

Any method described herein is intended to include within the scope of the embodiments of the present invention also any software or computer program performing all or any subset of the method's operations, including a mobile application, platform or operating system e.g. as stored in a medium, as well as combining the computer program with a hardware device to perform all or any subset of the operations of the method.

Data can be stored on one or more tangible or intangible computer readable media stored at one or more different locations, different network nodes or different storage devices at a single node or location.

It is appreciated that any computer data storage technology, including any type of storage or memory and any type of computer components and recording media that retain digital data used for computing for an interval of time, and any type of information retention technology, may be used to store the various data provided and employed herein. Suitable computer data storage or information retention apparatus may include apparatus which is primary, secondary, tertiary or off-line; which is of any type or level or amount or category of volatility, differentiation, mutability, accessibility, addressability, capacity, performance and energy use; and which is based on any suitable technologies such as semiconductor, magnetic, optical, paper and others.

It is appreciated that elements illustrated in more than one drawings, and/or elements separately referred to in the written description may still be combined into a single embodiment, except if otherwise specifically clarified herewithin.

### DETAILED DESCRIPTION OF CERTAIN EMBODIMENTS

The following terms may be construed either in accordance with any definition thereof appearing in the prior art literature or in accordance with the specification, or to include in their respective scopes, the following:

“Rotation” typically refers to or includes data describing a mobile device's rotation about each of or any subset of the x, y and z axes.

“Pattern” aka “repetitive pattern” typically refers to or includes a single end user stride aka gait cycle.

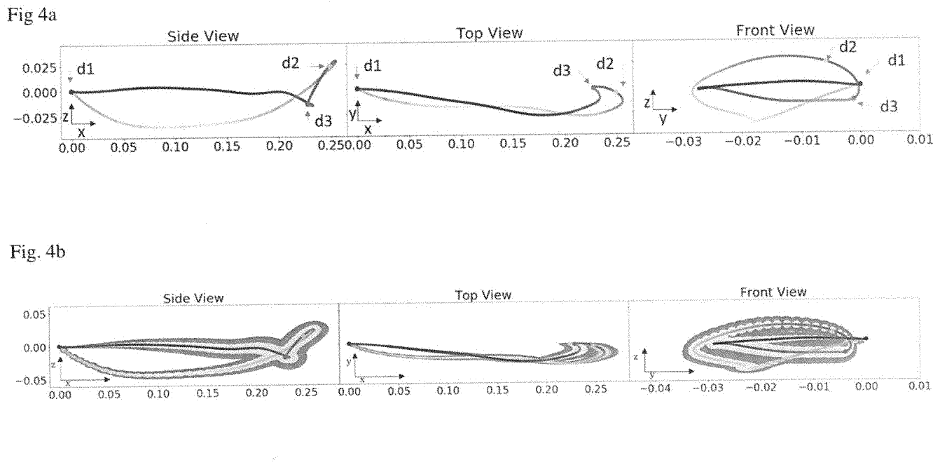

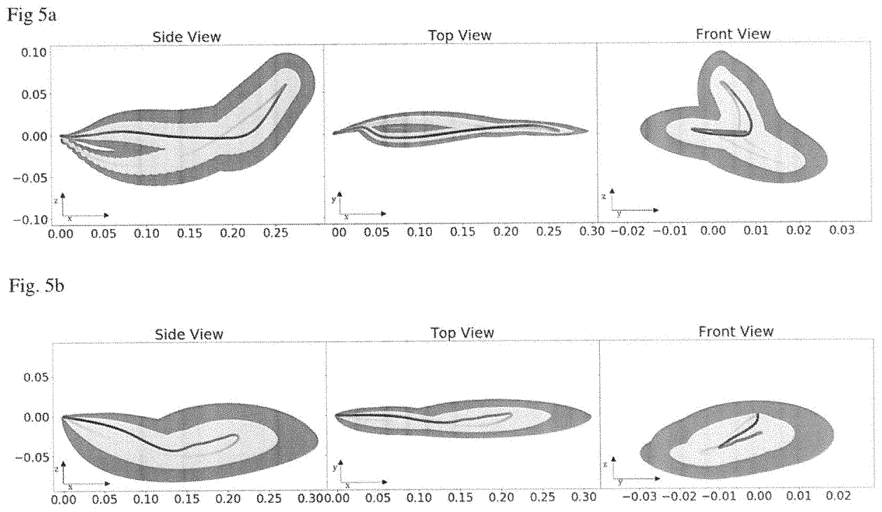

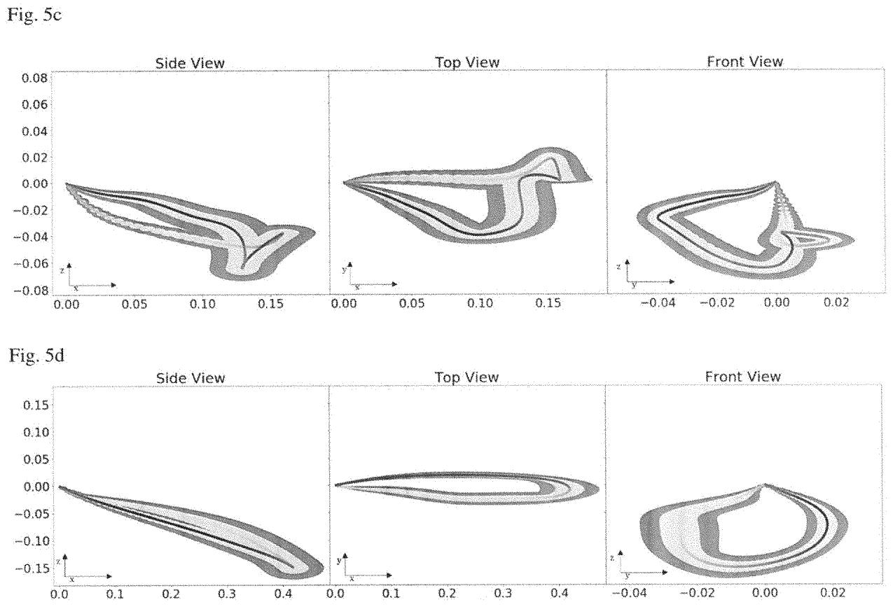

The closed-cycle graphs of, say, FIGS. 4a-5d each represent a given end-user's average “repetitive pattern”.

“Phase” typically refers to each of the conventional stages (typically numbering 100) that a stride includes. It is appreciated that some gait parameters, but not all, are phase-specific e.g. “duration of the swing phase” (the phase during which the foot is not in contact with the ground), or stance durations. In a system or operation mode of a system which derives parameters such as cadence and stride length which are not phase-specific, but does not derive any phase-specific parameters, it may be superfluous for the system to partition the stride into phases. However, typically, the system partitions or divides the closed-cycle graphs into phases which may respectively be indicated by 100 (typically) points along each graph.

“Baseline/s” typically refers to or determines expected values for a given user's gait parameters. Typically, a user's baseline is defined for each gait parameter p as the distribution of the values that parameter p assumes. Typically, the system constantly improves the estimation of p's baseline as more values of p are sampled or derived over time.

Modem smart devices, e.g. smartphones, boast a winning combination of motion sensors based on magneto-inertial technology and computation power, and are ubiquitous. Given the fact that end-users have their phones with them almost every day, almost all day long, this combination may be harnessed to provide a continuous gait monitoring system, including analysis. Gait is indicative of health, and is sometimes even more informative than other vital signs. Gait analysis can warn of express acute events such as stroke or injuries, trends of illness, or rehabilitation and recovery. Certain embodiments herein convert a smartphone into a gait monitoring system, for tracking gait trends.

Certain embodiments provide a wearable e.g. cellphone which analyzes the end user's gait including, say, determining in real time or near real time that an end-user is at risk for possibly experiencing a stroke which requires emergency treatment, and/or, say, providing biofeedback to an end-user who is doing physiotherapy or interested in self-help for improving or modifying her or his gait, for example making her or his gait more feminine or more masculine, for dating purposes.

Certain embodiments provide a software-only system that can be downloaded, e.g. as a cell app, onto any conventional cellphone.

Certain embodiments run continually, e.g. in the background.

Typically, the system need not receive external inputs identifying the device's bodily position and/or the system need not receive external inputs identifying the activity in which the end-user is currently engaged and/or the system's operation does not rely on an assumption that the device is constantly in a given bodily position or that the end-user is constantly engaging in a certain activity.

Typically, an algorithm for analyzing gait is selected, depending inter alia on whether the device is in the end-user's shirt pocket, or pants pocket, or hand, etc.

Typically, the system uses a classifier to determine the device's bodily position and/or the activity in which the end-user is currently engaged. The classifier may classify simultaneously along 2 dimensions: device position (in pocket/hand etc.)×user activity (walking/running/climbing stairs etc.).

Certain embodiments herein convert a smartphone into a gait monitoring system, for supporting recovery process.

Certain embodiments herein convert a smartphone into a gait monitoring system with real-time feedback.

Certain embodiments herein convert a smartphone into a gait monitoring system, for detection of acute events.

Certain embodiments provide all or any subset of the following advantages:
1. Continuous, gait monitoring
2. Gait monitoring at home
3. Based only on a smartphone; cost close to zero, due to the high availability of smartphones.
4. Passive, seamless. For example, the end user typically is not required to press any button, or otherwise activate the system, nor, typically, is the end-user required to hold the phone in any specific bodily position such as in her or his hand, nor is the end-user expected to indicate which activity s/he is engaged in.
5. Stride aka step detection at high accuracy and recall.
6. Gait analysis capable of measuring standard parameters measured by professional motion labs such as all or any subset of step and stride (left and right steps together), cadence speed and length, stance and swing durations, gait cycle phase detection as foot-contact and toe-off, hip (and maybe other joints) angle ranges, and angles, moments and power over the gait-cycle, and comparison of any of the above parameters between right and left legs. Typically, gait is analyzed and output indications of at least one gait parameter/s is/are provided in real-time or within seconds thereby enabling extremely prompt feedback and alerts.

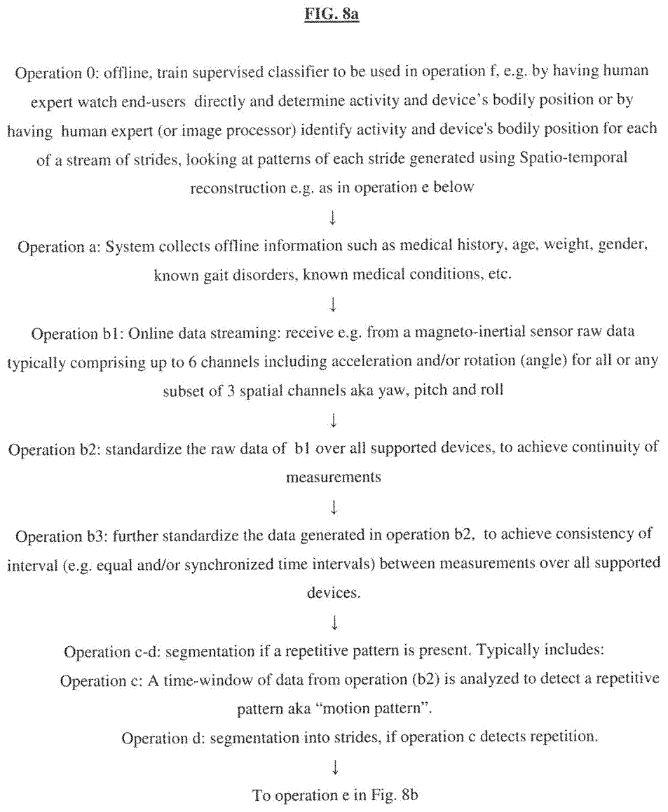

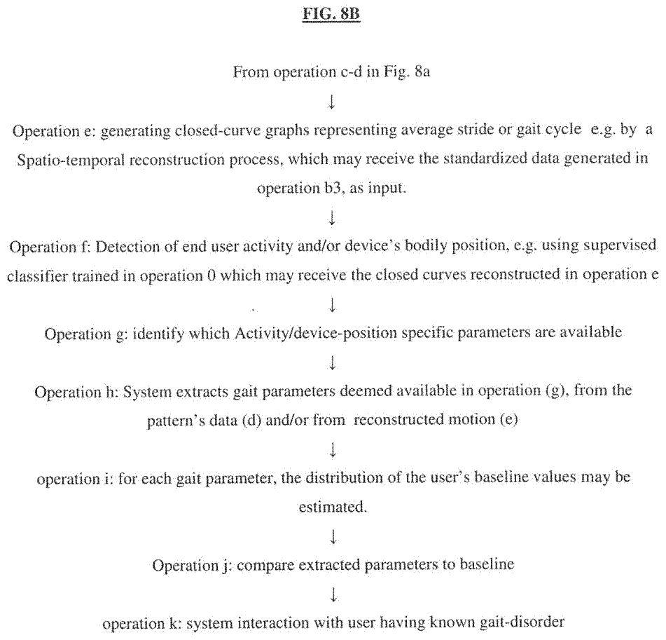

The main flow of FIGS. 8a-8b may include all or any subset of the following operations, e.g. as illustrated or as described hereinbelow, in any suitable order e.g. as shown:
Operation a: System collects offline information such as medical history, age, gender, etc. Typically, this information is collected during a registration process when the system/application is installed.
Operation b1—Online data streaming: receive from a magneto-inertial sensor raw acceleration and/or rotation (angle) for all or any subset of 3 spatial channels aka yaw, pitch and roll.
Operation b2: standardize the raw data received in operation bl over all supported devices, to achieve continuity of measurements.
Operation b3: further standardize the data generated in operation b2, to achieve consistency of intervals between measurements over all supported devices.
Operation c: A time-window of, say, the last few seconds of data from operation (b) is analyzed in an attempt to discern a repetitive pattern, aka “motion pattern”.
Operation d: If a repetitive pattern is found in (c) then data from (b) is segmented according to the pattern. Otherwise, the flow typically ends here.

According to certain embodiments, an “other” or stationary class (e.g. user is eating, or in car), is used by the activity classifier. Alternatively or in addition, the main flow includes logic which omits or skips operations g-k if user's activity is identified, in operation f, as being a stationary activity (such as eating, in car). Instead, the main flow waits until a record of a dynamic activity is encountered, from which it is meaningful for the system to extract gait parameters.
Operations c, d are sometimes referred to herein as a single operation, operation c-d, in which a repetitive pattern, if such a pattern exists, is segmented and otherwise the flow ends, e.g. for lack of any detectable repetitive motion.
Typically each segment represents a single cycle or a single stride.

Thus, according to certain embodiments, segmentation produces the objects (motion cycles/strides) for gait analysis. It is appreciated that any suitable processing of the objects, e.g. dosed-curve graphs, may be employed to extract parameters (or to detect activity/position pairs), not necessarily those specifically referred to herein. For example, suitable processing techniques may be “borrowed” from the field of image processing.
Operation e: Segmented data from (d) is used to reconstruct a spatial-temporal pattern of the motion aka movement.
Typically, each gait cycle is divided or partitioned into, say, 100 (or some other system constant number of) units aka phase units aka phases. The reconstruction process's output typically comprises the position (e.g. rotation and/or deviations) of the motion pattern at every phase unit relative to the user's movement, or to the inertial frame of reference. Thus the reconstructed positions are typically compared to this frame, which is useful since the reconstructed shape or object typically includes the mobile phone's position at every phase, and the rotation at every phase of the end-user's gait.
Operation f: Typically, the reconstructed motion or shape or object from operation (e), rather than the raw data, is used to identify the user's activity and device's position e.g. in user's (right/left) hand vs. in (right/left/hip/shirt) pocket.
Operation g: System deduces (e.g. retrieves from tables which may be stored in central system memory) which parameters are available given the activity and device position determined in operation f. Typically, the tables are constructed such that a parameter is deemed available, if a function can be defined that extracts that parameter from a pattern that represents the activity-position pair.
Operation h: System extracts parameters that are deemed available in operation (g), from the pattern's data (d) and reconstructed motion (e).
Operation i: Baseline for the user's specific activity and position is updated using the extracted parameters' values from (h) and from the activity-position detected on (f).
Operation j: The extracted parameters from (h) are compared to the baseline as most recently updated in operation i. If parameters' values from (h) differ from the user's baseline summarized over enough iterations of operation (i), system classifies the “gap”
aka difference between current gait parameters, and stored “baseline” parameters for that end-user, thus, typically, operation j detects abnormal gait for each end-user such that one end-user's abnormal gait may be the normal gait of another end-user.

It is appreciated that comparing an end-user's current gait characteristics with the same end-user's stored baseline is advantageous because this enables gait improvement or degeneration to be detected; rapid degeneration may for example be due to an emergency event.

Operation k—the system typically provides system/user interaction, e.g. each time the classification of (j) indicates a change, the system may provide user notifications e.g. via SMS or via Emergency SOS in iOS 11 (which triggers the iPhone to automatically call a local emergency number) or an audio notification to a user to initiate a stroke checkup (raise your hands, repeat a sentence, smile to the camera) or a short questionnaire: (why is your gait swinging, have you been drinking alcohol), etc. Another option is notifying the family e.g. parent of a minor or designated family member of a senior via SMS or the app push-notifications. based on the specific use case that a product e.g. app may handle. For example, a recovery product (e.g. app supporting physiotherapy for stroke or car accident survivors) may notify the user via SMS or via a daily or weekly summarizing email, of improvements s/he has achieved in her or his gait parameters. Or, a product that warns of risks may interact with the user, or directly with emergency services, when new pathologies are detected.

The flow of FIGS. 8a-8b may be performed at any suitable interval or on any suitable occasion. For example, the flow may be performed each time the raw data is sampled, or may be performed several dozen times per second.

Many variations are possible. For example, in contrast to certain embodiments described herein, the system need not necessarily compare an end-user's mobile phone's movement to an inertial frame of reference that moves with the end user's body and extract parameters on that basis. Instead, extraction of parameters and analysis of the segment may rely on, say, segmented raw data or some other preprocessing on the segmented data, or even may directly derive parameters from the raw data itself.

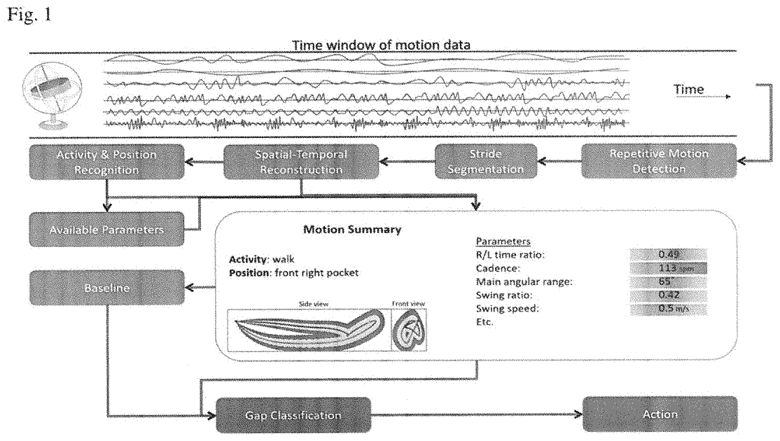

FIG. 1 illustrates logic blocks, all or any subset of which may be provided, each of which may be performed by suitable circuitry e.g. in software. The system of FIG. 1 may be used to perform all or any subset of the operations of FIGS. 8a-8b. For example, raw data recording may be followed by repetitive pattern detection (e.g. as per operation c herein), segmentation into strides (e.g. as per operation d herein), spatial-temporal reconstruction (e.g. as per operation e herein), activity and mobile bodily position recognition (e.g. as per operation f herein), and providing digital storage of and/or an output indication of a motion's summary including any characterization of gait or gait parameters shown and described herein. In some embodiments the system classifies deviation of the parameters' values from the baseline, and acts accordingly. For example, if the deviation is indicative of high stroke risk, the system may act to provide a near real time alert to emergency services.

An example implementation of the operation flow of FIGS. 8a-8b is now described.

First, gait parameter extraction, e.g. according to certain embodiments of operation h, is described generally. Parameters or statistics that describe the motion, including quantizing features such as but not limited to all or any subset of stride cadence, the duration of some gait cycle phases, such as when the foot touches the ground, the angles and speed at some points of interests, step length, step time (AKA stride length, stride time), gait velocity, presence/absence of a specific abnormality such as asymmetry, etc. Parameters include spatial parameters that describe the motion's shape, and temporal parameters that describe the motion's rhythm.

The system may, according to certain embodiments, define secondary or new parameters, as a function of the above. For example, parameter values extracted in different contexts of activity and device's bodily positions, may be compared, yielding new (aka “derived” or “secondary”) parameters. For example, to facilitate computation of asymmetry, which is an example of a derived or secondary parameter, the system may maintain pairs of parameters—front right pants pocket parameters and front left pocket parameters. A difference between corresponding left and right parameters means asymmetry.

For instance, parameters extracted from the gait of an end user bearing his device in his front left pocket may he compared to the gait of the same end user bearing his device in his front right pocket, to describe or characterize symmetry, or asymmetry between each leg.

It is appreciated that user x, Joe, may always put his phone in his right pocket, and never in his left pocket, which may, according to certain embodiments, cause all asymmetry parameters to be defined as unavailable for user Joe. Or, user Joe may be prompted or reminded by the system to sometimes carry his mobile device in his left pocket, in order to facilitate gait asymmetry analysis for Joe.

Parameters are typically intuitive, or easily interpreted such that the system's parameters (e.g. stride cadence, “main rotation” (which the system may define as the angle of the hip flexion-extension over the cycle) as a function of the gait phase, maximum speed of the thigh during swing) and motion representation are explainable to both professionals and end-users. Parameters typically correspond to standard descriptors of motion, such as magnitude of angles experienced by specific joints throughout the change of gait cycle phase e.g. as described in “Gait analysis—normal and pathological function.”, by Jacquelin Perry.

Parameters may be extracted from the spatio-temporal reconstructions generated in operation e. Parameters may be computed analytically, as functions right ahead of local measurements (such as velocity or rotation in some specific phase), and durations and spatial measurements between “events” (e.g. gait cycle phases, such as, say, Initial Contact, Loading Response, Terminal Swing).

Example: one parameter may be the point in time, within each stride, at which the opposite foot contact occurs (as is standard, the foot contact of the measured foot defines the 0 phase). Does opposite foot contact occur after, say, 55% of the cycle duration has elapsed, or after 60% or after 45%? For example, If a user's baseline indicates that for user x, opposite foot contact almost always occurs after 50-55% of the cycle duration has elapsed, then for user x, opposite foot contact which occurs late, e.g. after 60% of the cycle duration has elapsed, may be considered over-threshold, and trigger an abnormality alert.

Methods for identifying these events in the reconstruction are now described. It is appreciated that gait cycle phase durations such as, say, stance duration, swing duration, weight loading duration, are based on identifying the initial and final specific phase points.

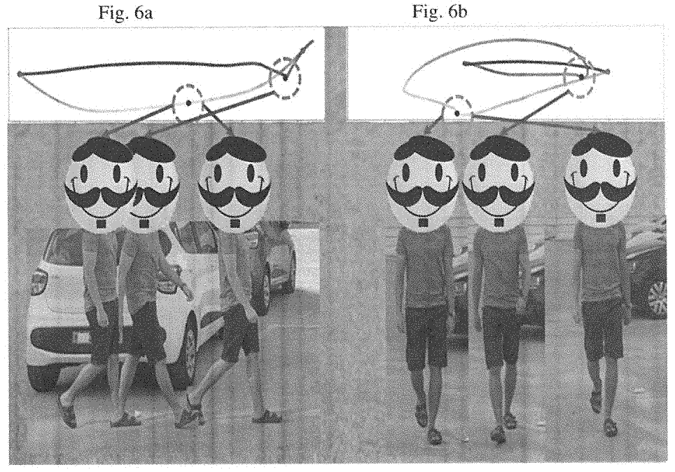

FIGS. 6a and 6b each illustrate a reconstruction of a walking end-user's single stride atop aligned videos of the walking end-user, whose smartphone is carried in the right front pocket. Since only a single stride is shown, no standard deviation is presented. In other cases, where more than one cycle is presented, the cycles may be summarized to yield mean motion or any suitable central tendency of any aspect of the end user's motion.

FIGS. 6a, 6b illustrate side and front views, respectively. Two points of interest are indicated, using dashed-line circles, within each of the reconstructions. The first point of interest, which is, in both FIGS. 6a and 6b, on the left, is a toe-off event, which can be identified as a point of change in motion direction, in the front view of FIG. 5b. The second point of interest, on the right in both FIGS. 6a and 6b, is a heel-strike event, which can be identified as a point of change in motion direction, in the side view of FIG. 6a.

Below, with particular reference to FIGS. 8a-8b, operation b is described, in which raw data is, e.g. continuously, provided by the magneto-inertial sensor, and how to standardize it is outlined. In operation c, a repetitive pattern may be detected in a time-window of standardized data, and operation d may generate the segmentation of the pattern over it. Operation e typically reconstructs an approximation of the motion in space and gait cycle phase, and some motion reconstructions are described below. Operation f typically comprises a process of identifying the user's activity and the device's position based on the segmented data and the motion reconstruction; all or any subset of the operations may be employed. in operation g, various activities and positions may determine which parameters are available, and may, therefore, be extracted. Motion/gait disorders can then be detected e.g. based on the motion reconstruction and its parameters, and new, aka secondary parameters, may then become relevant and may be extracted. A baseline may be defined for each user and suitable metrics may be used for evaluation of the user's gate quality. An example metric may be the likelihood of observable parameters' values determined by comparison of the observable values to the user's baseline. Any suitable scheme may determine when and how user interaction may be initiated e.g. as described herein. The system may integrate sensor timing to be more accurate and more efficient, e.g. as described herein.

### Operation b: Raw Data and Standardization

Raw motion data is typically provided by the device's motion sensors e.g. all or any subset of the wearable device's accelerometer/s, gyroscope:/s, gravity sensor/s, and magnetometer sensor/s. Most smartphones from a variety of manufacturers have all or a subset of the above sensors.

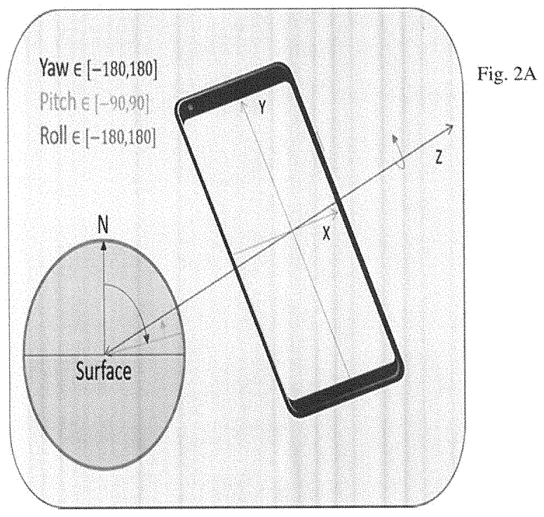

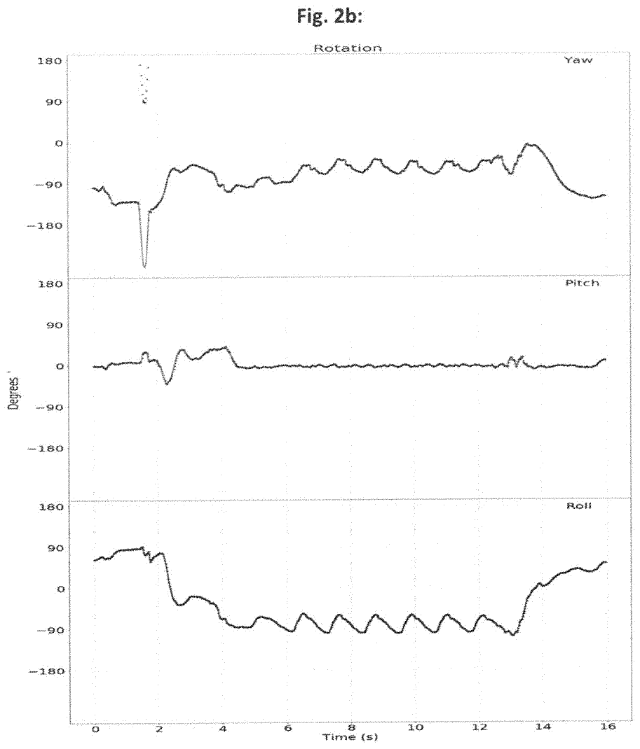

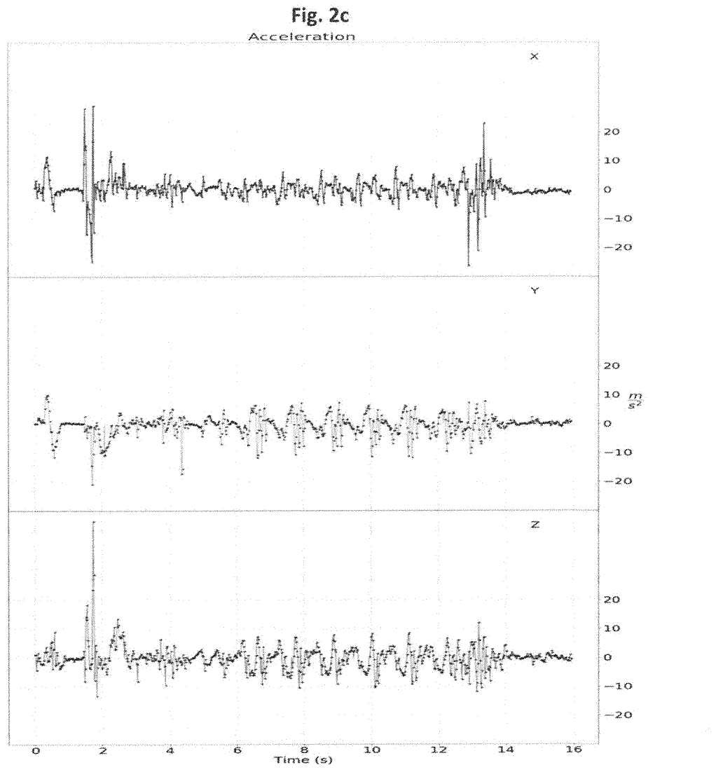

In FIG. 2A raw data describing the device's motion includes (all or any subset of) six different measures—three accelerations, e.g. measured relative to the device's frame of reference, and three angles between, say, the device's frame of reference and the earth's frame of reference.

Thus the raw data may include 6 channels comprising 3 channels of rotation in yaw pitch and roll, and 3 channels of acceleration according to each of the device's 3 axes. An example of a data record of a 16 second time window is shown in FIGS. 2B-2c.

In order to facilitate performance of a single process, for all supported devices, standardization or normalization over all devices is typically provided. For example, operations b2 and b3 respectively standardize the raw data in two fashions: continuity of the measurements, and consistency of intervals between measurements.

Regarding operation b2, conventional sensors often produce rotation angles in a bounded range (for example, the yaw angle is between −180 to 180 degrees), so when the rotation exceeds the range, the rotation starts over. For example, as soon as the yaw rotation angle exceeds +180 degrees, the yaw rotation angle reverts to −180 degrees. Operation b2 reverses this operation to yield data which, as in the real world, is continuous. For example, as shown in the upper left (yaw/rotation) graph of FIG. 2B, the yaw angle drops down and exceeds the value range; note the difference between the “fixed” raw data in dots seen in the upper left graph, where the angle exceeds the [−180, 180] range and the standardized reversed data which is a continuous line. More generally, FIGS. 2B and 2c illustrate an example measurement result showing the six different measurements of FIG. 2A which characterize the motion of a device which, in the example, was borne by an end-user who was walking. The continuous line shows the standardized data generated by operation b2, whereas the dots referred to above, in the upper (yaw/rotation) graph of FIG. 2b, denote the original raw data produced by the sensors. In the upper (rotation/yaw) graph, the effect of reversing the angle bounding is apparent, as the raw data differs from the standardized data. In addition, the diagram shows that the interpolation fits, hence represents, the data well (e.g. many most or all dots (the raw data) fall along the interpolated line).

Motion sensors typically have a predetermined uniform sampling rate, in theory. However, in practice, their sampling points of time are not exact. Converting the raw data into consistent intervals, i.e. a sequence of values (which may be raw data values sampled directly by the device's magneto-inertial sensor) in fixed points of time spaced equally in a. specific rate, typically comprises interpolation (which may be linear) of the data in time. Fortunately, it turns out that conventional sensors' sampling rates (approximately 100 samples per second) are higher than needed. Operation b2 typically converts the conventional sensors' sampling rates to a system-wide uniform or consistent rate of 50 samples per second, which is still enough to ensure that, as can be seen in FIGS. 2B-2c, the raw data fit the standardized data very well. According to certain embodiments, conventional interpolation methods may be used to fit the data, such as, say, linear interpolation.

Operation c-d: Segmentation into Strides

Many methods for stride detection are known; acceleration data is typically segmented along the time axis, of into strides. Methods for performing such segmentation are described e.g. in:

Perez, Andres A., and Miguel A. Labrador. “A smartphone-based system for clinical gait assessment.” 2016 IEEE International Conference on Smart Computing (SMARTCOMP). IEEE, 2016; and/or

Gadaleta, Matteo, and Michele Rossi. “Idnet: Smartphone-based gait recognition with convolutional neural networks.” Pattern Recognition 74 (2018): 25-37. Alternatively, rotation data may be segmented, along the time-axis, into strides. Any stride detection technique may be employed. Described herein is an example of a heuristic method to extract segments of strides from rotation data; alternatively however, the methods of Perez/Labrador or Gadaleta/Rossi may be employed.

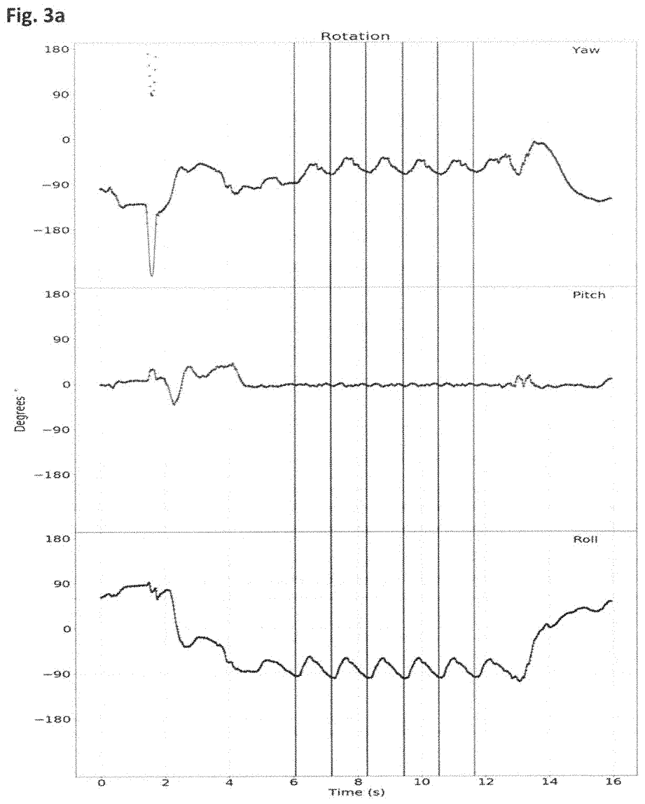

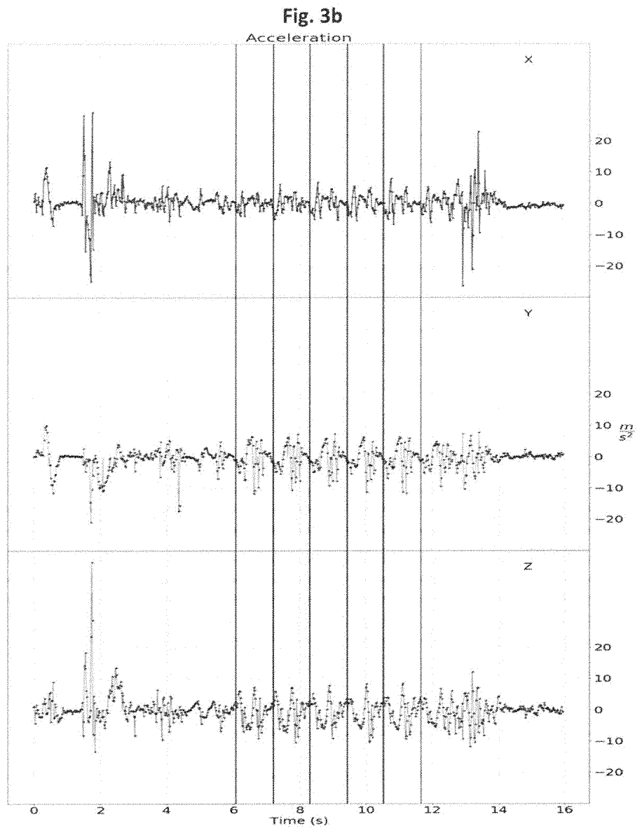

An example of stride segmentation results generated from the motion record rotation data in FIG. 2b, is shown in FIGS. 3a-3b.

Example Pseudo-code for segmentation into strides; all or any subset of lines of the following code may be used:

| Let Acc ∈ [symbol] ^{3Xn} be the acceleration data | |
|---|---|
| Let Rot ∈ [symbol] ^{3Xn} be the rotation data | |
| | //Check if the motion record is not static |
| 1. | velocity = cumsum(Acc, axis=1) |
| 2. | energy = sum(sum(velocity**2, axis=0), axis=1) |
| 3. | if(energy < energy_threshold): |
| | 3.1. return None |
| | //Ignore the yaw component of Rot |
| 4. | smooth_Rot = Rot[1:,:] |
| | //Make rotation smoother with convolution, mask size is 0.25 |
| | second in sampling rate unit |
| 5. | smooth_Rot = conv(smooth_Rot, Gaussian1D(0.25 * freq)) |
| | //Take the main component of rotation deviation |
| 6. | smooth_Rot = PCA.fit_transform(smooth_Rot)[0,:] |
| | //Finding extremum point is straight forward |
| 7. | extremum_indexes = find_extremum_points(smooth_Rot) |
| 8. | extremum_indexes_a = extremum_indexes[::2] |
| 9. | extremum_indexes_b = extremum_indexes[1::2] |
| 10. | range_a = std(smooth_Rot[extremum_indexes_a]) |
| 11. | range_b = std(smooth_Rot[extremum_indexes_b]) |
| 12. | if(range_a > range_threshold OR range_b > range_threshold) |
| | 12.1. return None |
| 13. | duration_a = std(diff(extremum_indexes_a)) |
| 14. | duration_b = std(diff(extremum_indexes_b)) |
| 15. | if(duration_a > duration_threshold OR duration_b > |
| | duration_threshold ) |
| | 15.1. return None |
| 16. | strong_forces =sum(Acc**2, axis=0) |
| | //Sum forces locally (0.25 second) |
| 17. | strong_forces = conv(strong_forces , Rect(0.25 * freq)) |
| 18. | sum_force_a = sum(strong_forces[extrernum_indexes_a]) |
| 19. | sum_force_b = sum(strong_forces[extremum_indexes_b]) |
| 20. | if(sum_force_a > sum_force_b) |
| | 20.1. return extremum_indexes_a |
| 21. | return extremum_indexes_b |

### Operation e: Spatio-Temporal Reconstruction

As described above, each gait cycle is divided or partitioned into 100 units aka phase units aka phases. 100 is the standard parameter used in gait analysis literature although, alternatively, another typically system-constant number of phases may be used. The reconstruction process's output typically comprises the position (rotation and deviations) of the motion pattern at every phase unit relative to the user's movement.

Reconstruction of the motion in space by gait cycle phase typically yields a cycle including 100 points in time that divide the cycle into 100 equal time-intervals.

These intervals and points or dots are useful both for visualization of the pattern, but also for estimation of the characteristic pattern of the user's activity relative to the position. For example, the system may summarize different motion records as one characteristic pattern, which provides information regarding the user's baseline patterns of movement which may be stored as part of the baseline. It is appreciated that the entire reconstructed pattern (closed graph) may be regarded as a parameter.

To provide alignment between different records, both rotation (e.g. of the mobile device about each of the x, y and z axes) and gait cycle phase, are typically taken into account or normalized during reconstruction of the closed graph. Otherwise, the resulting closed shapes each associated with a different stride in the record would, undesirably, be rotated or oriented differently in space.

To overcome the fact that different records are collected at different times, in which the device is in different orientations, rotation is typically applied to standardize or normalize the data. In the reconstruction operation e this yields standardized input for the next operation f, which is the closed graph (aka shape) with standard orientation. The target orientation may comprise:
i. the vertical direction toward the sky, which may be extracted directly from the rotation data e.g. assume that during gait, the device pivots or rotates about the vertical axis, like a pendulum. Thus, the vertical direction is derived by computing the average rotation, over a cycle segment/stride, of the pitch and roll angles. For example, if the average pitch rotation angle is x and the average roll rotation angle is y, the vertical direction may be computed by reversing the rotation of the roll (rotate the roll with −y) and then reversing the rotation of the pitch referenced to the vertical direction (rotate 90−x).
ii. the main displacement of the movement, which is orthogonal to the vertical), which can be extracted directly (by double integration e.g.) from the acceleration data; and
iii. the third direction, that is orthogonal to both the above, which represents horizontal movement.

The term “main displacement” is intended to include the first component of principal component analysis (PCA), or the displacement of the most distanced point from the beginning or other edge of the closed figure or graph, both of these typically yielding similar results.

Gait cycle phase: Typically, when the system extracts the segmented stride data the system typically (in segmentation operation c-d) consistently starts the cut or segmentation (thereby defining the starting point of each segment) of all strides from all records at the same gait phase e.g. the extremum point of the rotation the starting points of the segmentations. The term “rotation” according to certain embodiments, is intended to include rotation or pendelum-like motion of a mobile device within a vertical plane, as a person strides.

Typically, in segmentation operation c-d, the rotation is the first principal component analysis (PCA) component of the pitch and the roll angles, and the system marks segmentations according to the peaks of this component over time.

More generally, operation c-d typically consistently starts segmentation in a specific phase of cycle (e.g. always starts segmenting from the same phase).

Typically, the gait cycle phase is both consistent and describes a phase relative to a clearly defined temporal reference point. That temporal reference point is typically the point in time, from which to start integration of acceleration to velocity, and velocity to displacement. Typically, the temporal reference point is selected to be a point of change in the movement direction. The two candidates for temporal reference point are typically the two extremum points of rotation. Typically, from among the two extremum points of rotation, the rearmost one (from the movement direction point of view) is selected. The rearmost extremum point of rotation is also typically the point in time at which a new segment begins.

Typically, the reconstruction is relative to (e.g. factors out) the motion of the end user's body as a whole (which is typically similar to the motion of the end user's body's center of mass). The average speed during the stride is typically subtracted when the system closes the shape, because the displacement summarizes to 0.

Consider the pocket of a person walking on a treadmill; when this person ends a stride, his pocket returns to its initial position, hence his gait cycle completes a closed shape. In contrast, in any other stationary reference system, the gait cycle, unless the end user's entire body motion is factored out, the gait cycle would not complete a closed shape. The reconstruction typically generates or creates or completes a cycle that ends at the same point as the reconstructed shape, or from which the cycle starts. To verify that the reconstruction of the displacement completes a cycle, the logic herein typically is configured to ensure that the double integration of the velocity over each segmentation, or the double integration of the acceleration data over each segmentation, sums to zero e.g. by subtracting the average over the cycle interval velocity from the momentary velocity, which is tantamount to making the typically reasonable approximation that the end user's body (or center of mass thereof) moves at a constant speed during a single stride.

Typically, plural segments from the same record are aggregated, e.g. by averaging, into one pattern of gait/motion cycle (one closed graph e.g. as shown in FIGS. 4a-5d). For example, the reconstruction of FIGS. 4a, 4b happens to be an average pattern taken over five strides, although this is not intended to be limiting.

Typically, the shapes (displacement and rotation over the cycle phases, each typically including 100 points) described herein are averaged to yield a pattern cycle. Typically, the system is operative to average the motion cycles at each point and store the standard deviation. The resulting averaged pattern is used as the reconstructed pattern (aka shape) of the record.

Similarity of a current gait of end user x to, or deviation from, x's baseline may then be determined by parameters' values extracted from the above reconstructed shapes.

FIG. 4b is a graph of an example reconstruction aka closed graph aka shape, of a stride taken by a person whose device is, fixed with respect to the thigh, e.g. is positioned in the right front (say) pocket of the end-user's jeans. As is apparent from FIG. 4b, it is typically safe to assume that such a device's position remains fixed with respect to the thigh, throughout the stride, with the exception of some tilts along axes orthogonal to the end user's direction of motion whose magnitudes are not significant compared to the magnitude of the end user's motion along his direction of movement (e.g. north, if the user is walking northward).

FIG. 4b includes:

(a.) A side-view—shows the vertical dimension and the dimension of the movement direction, whereas the horizontal dimension is orthogonal to the plane of the page and therefore cannot be seen.

FIG. 4a includes:
(a.) A Side-View—shows the dimension of the movement direction (x) and the vertical dimension (z). Again, the horizontal dimension (y) is orthogonal to the plane of the page, hence cannot be seen.

FIGS. 4, 4a both also include:

(b.) Top-View—shows the horizontal dimension and the dimension of the movement direction, whereas the vertical dimension is orthogonal to the plane of the page, and therefore cannot be seen;

(c.) Front-View—shows the horizontal and vertical dimensions; the movement dimension is orthogonal to the plane of the page and therefore cannot be seen. In the side- and top views, the aspect-ratio of the axes is equal, whereas in the front-view it is not; instead, the dimension proportion or aspect ratio of the front view is stretched so the motion can be better appreciated, or tracked more easily.

It is appreciated that the closed-curve graphs shown and described herein are but one output that may be generated by the system shown and described herein. Alternatively or in addition, other outputs e.g. graphs may be generated, such as any tabular or graph or alphanumeric or verbal representation or description of a given individual's (average) gait, or of certain aspects thereof, which are conventionally generated by known gait analysis systems.

Three dots are illustrated in the graphs of FIGS. 4b and 4a; they are marked d1, d2, d3 in the graph of FIG. 4a. The darkest one, d1, (the leftmost one in the Side-View) indicates the start of integration and the first change of the movement direction; the lightest one, d2, indicates the second change of the movement direction; and the third dot, d3, indicates the point when the heel touches the ground.

The line shown in FIGS. 4, 4a (aka average line) represents the locations, during the stride (typically, the average locations at each time-point during the stride, average over several e.g. 5 strides), of the device's center (of the point halfway between the device's top and bottom edges and halfway between the device's right and left edges).

Three dots d1, d2, d3 are illustrated in the graphs of FIG. 4a. d1 (the leftmost dot in the Side-View) indicates the start of integration (of velocity and displacement) and the first change of the movement direction. d2 indicates the second change of the movement direction; the third dot, d3, indicates the point when the heel touches the ground.

The reconstruction of FIGS. 4b, 4a is an average pattern of 5 strides; the variance in the device center's location at a given time relative to the starting point of the stride, is represented by a light grey area surrounding the line which represents all points which are up to half a standard deviation from the line, and by a dark gray area which the geometric location of all points in space which are more than 0.5 standard deviations and less than 1 standard deviation, from the line.

The brightness of the line, in FIG. 4a, represents the speed of the motion in the movement direction, typically relative to the “speed of the center of mass”.

Light color indicates forward, and darker indicates backward. The brightness of the line, in FIG. 4a, also represents the speed of motion in the movement direction: light color indicates forward and darker indicates backward.

This is typically illustrated to show the direction of the motion or to indicate whether the motion is clockwise or counterclockwise.

The reconstructions Illustrated in FIGS. 4 and 4a and in other drawings herein show only displacement and do not show rotation along with gait cycle phase, however, the mean rotation's magnitude, direction and/or standard deviation are also typically determined or computed or derived, as part of reconstruction operation e. The reconstruction process typically includes aggregation, e.g. as described herein, of plural cycles (e.g. for all strides extracted from the record).

Since reconstruction is a preprocessing stage for the activity-position classification operation and/or the parameter extraction operation, each or both of which operations may use all or any data provided as a reconstructed pattern of cycle of a certain record (e.g. mean rotation's magnitude, direction and/or standard deviation).

Operation f: Detection of End User Activity and Device's Bodily Position

Activity (such as running, jogging, walk and stairs climbing, and device's bodily position (e.g. in pocket, hand-held) are detected then used to optimize analysis of such motion. This way, no matter what kind of activity the user is doing, or the position the device is being carried, the system may assess the user's baseline and identify disorders and irregularities relative to that specific activity-device's bodily position pair. Because it is possible to extract different parameters in different contexts of activity-position pairs, it is required to be more specific in definitions of activities and device's bodily position. For example, the system may distinguish between climbing up the stairs and going down. For the device's bodily position, the system may detect also the side of the position, for instance, when the position is the front pocket of the user's jeans or when the device is held by the hand.

There are a number of methods for recognition of a mobile device's end user's activity using his mobile device's acceleration and the rotation data directly, some of which are based on Deep Learning such as, say:

Murad, Abdulmajid, and Jae-Young Pyun. “Deep recurrent neural networks for human activity recognition.” Sensors 17.11 (2017): 2556.

Ordóñez, Francisco, and Daniel Roggen. “Deep convolutional and LSTM recurrent neural networks for multimodal wearable activity recognition.” Sensors 16.1 (2016): 115.

Others use segmented data for user identification rather than for activity identification, such as, say, Gadaleta, Matteo, and Michele Rossi. “Idnet: Smartphone-based gait recognition with convolutional neural networks.” Pattern Recognition 74 (2018): 25-37.

A particular advantage of certain embodiments is that the extracted pattern (e.g. as found in operation c) preserves most of the relevant information about the activity and the device's bodily position, and thus the rest of the data, e.g. the raw data, can be ignored. According to certain embodiments, the original or raw motion data may be approximately reconstructed by concatenating the pattern.

As described herein, the system typically but not necessarily uses the reconstructed patterns described herein as input for the activity-position classification task, rather than using the raw data directly.

Typically, speed affects the pattern across the walk, and thus various segments may be used to represent the user's same activity-device's bodily position context. Nonetheless, the reconstructed pattern, in practice, is found to contain substantially all useful information regarding the activity and the position that was originally included in the raw data, such that processing the reconstructed data, rather than the raw data, has justification. According to certain embodiments, the system may regenerate the raw data by concatenating the pattern according to the “strides'” or segmentations' positions, and stretching the strides or segmentations according to the “strides” durations, in order to reduce the effect that speed has. Therefore, the system loses less information and the reconstructed pattern preserves almost all information present in the raw data.

It is appreciated that according to certain embodiments, the pattern is normalized such that the speed doesn't change the pattern (in contrast, the same activity and position appear different each time the end-user's gait speed changes, if a raw pattern, rather than the pattern as reconstructed herein, is viewed (or analyzed). The reconstructed pattern is typically independent of the speed as long as the activity remains similar (as opposed to walking which becomes running, in which case the gait cycle and thus gait parameters do change). If the activity does not change (e.g. in the case of fast vs. slow walking, but not running) the speed is just the translation of a phase unit into a time duration.

References herein to computation of device motion relative to end-user's center of mass, refer not to actual speed of the end-user's center of mass, which is not directly known, but instead to the average speed of the mobile device during the cycle (which approximates the “speed of the center of mass” since if the speed of the center of mass does not change during the cycle, then these two are indeed the same). Alternatively, a central tendency other than the average speed may be used to approximate the “speed of the center of mass” e.g. a mode or median value or a suitable logical combination of the mobile device's average and/or mode and/or median speeds, over the cycle.

The method herein, unlike conventional methods, uses the reconstructed spatio-temporal pattern, generated in operation e, which typically computes (e.g. by averaging speed over the cycle) the motion of the device relative to the center of body speed e.g. velocity of the end user's body's center of mass by the gait cycle phase. This approach is more robust against changes in the end-user's speed of motion (e.g. if the end-user starts walking more slowly, or conversely starts running) because the reconstructed pattern depends on the phase and is independent of the speed, and looks similar in different speeds.

To classify the activity-device's bodily position pair, operation f may use supervised DL (deep learning) techniques based on a supervised classifier having a multi-class classification architecture composed of CNN (convolutional neural networks) layers. The top layer gets the fixed-sized reconstructed pattern, generated by operation e, as input.

Alternatively, activity and device bodily position may each be recognized independently, rather than recognizing pairs of activities and bodily positions. Also, the dimensions of device bodily position may each be recognized independently e.g. recognize whether the device is on the right or on the left, independently of whether the device is in a pocket, or handheld, or strapped to the arm.

Activity/position pairs which are used may include all or any subset of the following:

WALKING, RIGHT BACK HIP POCKET OF TIGHT PANTS

WALKING, LEFT BACK HIP POCKET OF TIGHT PANTS

WALKING, RIGHT FRONT HIP POCKET OF TIGHT PANTS

WALKING, LEFT FRONT HIP POCKET OF TIGHT PANTS

WALKING, RIGHT SHIRT POCKET

WALKING, LEFT SHIRT POCKET

WALKING, HELD IN RIGHT HAND

WALKING, HELD IN LEFT HAND

8 MORE PAIRS, AS ABOVE BUT FOR GOING UPSTAIRS RATHER THAN FOR WALKING.

8 MORE PAIRS, AS ABOVE BUT FOR GOING DOWNSTAIRS,

8 MORE PAIRS, AS ABOVE BUT FOR RUNNING

OTHER (an “other” class may be provided, or an “other” position may be defined, to include all positions other than those listed above e.g. “IN SKIRT”, “in loose pants” “IN HANDBAG”, “IN BACKPACK”, “IN BABY CARRIAGE”.

In addition, the spatio-temporal reconstruction of operation e, when represented visually e.g. as in FIGS. 4 and 5a-5d, allow a human expert (or image processing functionality) to recognize activity-device's bodily position pairs as may be appreciated by comparing FIGS. 5a-5d (and FIG. 4b). For example, a human expert or image processing logic can receive an incoming reconstruction and determine whether the incoming reconstruction is most similar to the reconstruction of FIG. 4b, to the reconstruction of FIG. 5a, to that of FIG. 5b, or to that of FIG. 5c, or to that of FIG. 5d, or to the reconstructions of whichever other activity-device's bodily position pairs are being used by the system.

This allows labels to be efficiently gathered for training the supervised classifier used in operation f, without requiring the human expert to actually determine the activity-device's bodily position pair by watching the user during his activity. Instead, the human expert (or image processor) identifies this pair efficiently, for each of a stream of strides, looking at the reconstructed pattern of the stride.

For example, still referring to the reconstructions of various activities/device's bodily positions pairs shown by way of example in FIGS. 5a-5d, it is appreciated that when the bearer of the mobile device, or end-user, climbs stairs (FIG. 5a), since the device is fixed, generally, relative to the thigh, the swing that starts the motion gets relatively high, followed by mainly vertical movement to lift the end-user's body up to the next step in the flight of stairs, and so the thigh gets lower. When going downstairs (FIG. 5b), the swing ends lower compared to upstairs FIG. 5a. The difference in the device's bodily position between the two motions can be distinguished by the convex shape, as can be seen from the top view in FIGS. 5a vs. 5b: the left front pocket position looks convex relative to the right direction (down—note that when looking at the user's movement from the top point of view and assuming forward is the x axis (to the right), motion to the left is up, on the page, whereas right is down), and the right front pocket position looks convex relative to the left direction (up). The difference is also apparent when the front views of FIGS. 5a, 5b are compared, as the movement ends; (the motion starts at 0,0(,0), proceeds with the light line and ends as the line becomes darker) with a V-like shape, when the V opens to the left as in FIG. 5a, the device is in the left pocket and vice-versa, if the V opens to the right (not shown) the device is in the right pocket. The device-in-hand position of FIG. 5d can be easily recognized as having no points of discontinuity thus no noticeable features and thus may be deemed to have a dominant component. In the movement direction (e.g. the range of motion along the axis along which the end user is moving, aka main axis or axis of movement, is much larger than the range of motion along the two other axes. The back pocket position (e.g. as shown in FIG. 5c) also is recognizable, also due to its own unique features, e.g. as shown in the middle graph of FIG. 5c, the first half of the cycle (characterized by movement forward) has a much smaller horizontal component, than second half of the cycle, characterized by movement backwards.

Operation g: Identify Activity/Device-Position Specific Parameters

Some parameters, such as stride duration and cadence, are not specific to a particular activity of device position; they can be extracted from various device's bodily positions, others, however, are more specific. For example, the speed of the thigh during the swing requires a record of a walk from the front pants pocket i.e. is specific to the walk/front pants pocket class. In general, riot all parameters are accessible from any record regardless of the activity being done or the position the device was being carried in while that record was made, thus the activity and the position determine, to no small extent, which parameters to extract (and/or how to do so).

Typically, activity-position specific parameters and non-specific parameters are both handled similarly; the non-specific parameters are available in all or many activity-positions.

A table, which may be predefined, may be stored, to enable the system to look up which parameters are accessible for every activity and position. For each parameter the table may define computational functions that extract that parameter from records collected in different activity-position contexts. A certain function can be defined, or is applicable or relevant or available for only some situational data (for some activities but not others and/or for some device positions on the body and not others). In this case, e.g. for a certain activity-position pair (e.g. because the records or raw data that are needed as input to the function, are available for that pair) the table may indicate that this parameter (say: thigh rotation) is available (e.g. can be computed) for that pair, and may store the function to extract that parameter. For example, the maximal rotation of the thigh during the swing can typically be extracted when the device is in the end-user's front pocket and not when the device is in the end-user's hand. The function e.g. formula for each parameter, is typically derived straightforwardly from the parameter's definition.

During a period of time, an end-user performs various activities and holds his device in various positions. This typically facilitates a rich collection of parameters, yielding a very detailed representation of the user's motion. This means that the system takes advantage of the variety of activity-position contexts, rather than treating that variety as an impediment or difficulty.

It is appreciated that certain parameters may be applicable or relevant for all/many activities/positions pairs, but parameters normal range may differ, often mainly as a function of the activity, rather than, or more than, as a function of the device position. The system typically stores, and may update over time, a baseline for each parameter, either non-specific, or specific to, say, each of various activities for which the parameter is available.

For example, stride cadence may differ over activities, however, as long as the position in which the device is carried enables the cadence to be measured, the value is typically irrespective of position.

### Operation h: Gait Parameter Extraction

Parameters or statistics that describe the motion including quantizing features such as but not limited to all or any subset of stride cadence, the duration of some gait cycle phases like when the foot touches the ground, the angles e.g. between certain joints and the ground, or between themselves, and speed at some points of interest e.g. speed of the thigh for example, at the start/peak/end of the swing, at foot contact, etc.

Parameters include spatial parameters that describe the motion's shape, and temporal parameters that describe the motion's rhythm.

In addition, secondary or new parameters may e defined, as a function of the above. For example, parameter values extracted in different contexts of activity and device's bodily positions may be compared, yielding new (aka “derived” or “secondary” parameters. For instance, parameters extracted from the gait of an end user bearing his device in his front left pocket may be compared to the gait of an end user bearing his device in his front right pocket, to describe or characterize symmetry or asymmetry between the legs. Typically, the system computes lateral pairs of gait parameters e.g. a given gait parameter which characterizes end-user E's gait (possibly when E is engaged in activity A) when E's wearable is in E's front right pants pocket and the same parameter when E's wearable is in the front left pocket. Then, differences between corresponding/paired left and right parameters may be computed to quantify gait asymmetry.

Parameters (e.g. stride cadence, main rotation (e.g. flexion-extension rotation (or angle) of the hip), maximum speed of thigh during swing) are typically intuitive, or easily interpreted, such that the system's parameters and motion representation are explainable to both professionals and end-users. Main rotation may be computed as a function of the gait phase e.g. may be computed for each phase separately, since, as described herein, each reconstructed pattern typically includes 100 phase units, each having has its own parameters such as position, rotation, etc.

Parameters typically correspond to standard descriptors of motion such as magnitudes of angles experienced by studied joints as the gait cycle proceeds i.e. as the gate cycle phase changes. e.g. as described in “Gait analysis—normal and pathological function” by Jacquelin Perry.

In operation h, parameters may be extracted from the spatio-temporal reconstructions generated in operation e. Generally, parameters are computed, using the parameter's function e.g. formula as stored, of local (e.g. pertaining to a specific phase/point in the cycle pattern rather than to the entire cycle) measurements, such as velocity or rotation in some specific phase. Typically, these are computed from the reconstructed pattern, using a parameter-specific function whose inputs are the pattern's values around some phase. Durations and spatial measurements may be computed between “events” (e.g. between gait cycle phases, such as, say, Initial Contact, Loading Response, . . . Terminal Swing).

Example: IC˜0%, LR/opposite swing˜15%, Opposite Foot Contact˜50%, OLR/Swing Start˜65%. In this example the swing duration is 35%=the duration in the cycle between the starting phase of the swing and the initial contact.

It is appreciated that parameters may include the durations of various gait cycle phases, determined by computing the difference between that phase's initial and final phase points (e.g. between the phase's starting point and end-point, in time).

Methods for identifying events (e.g. gait cycle phases, such as, say, Initial Contact, Loading Response, . . . Terminal Swing) in the reconstruction e.g. for partitioning a given reconstruction into gait cycle phase (such as stance, swing, weight loading), are now described.

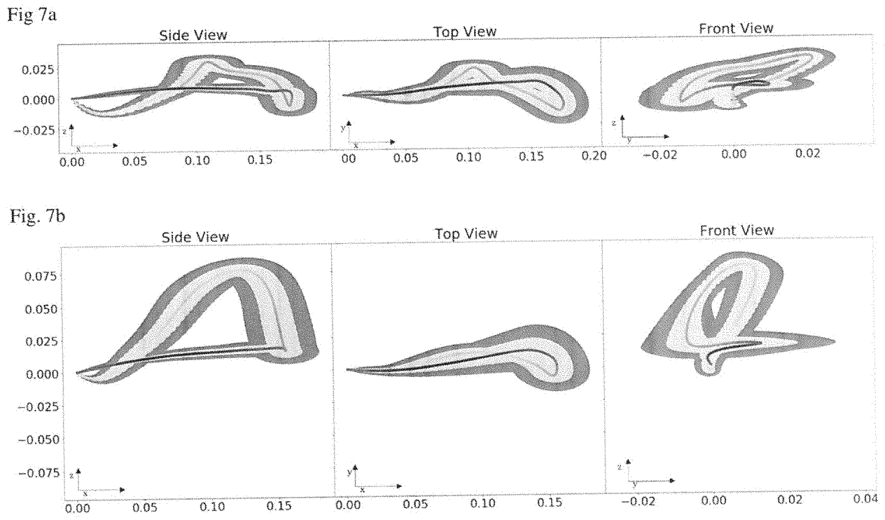

Detection of abnormal gait: Disorders, or abnormal gait, are defined patterns of motions or parameter values that differ from the standard, such as a limp which is asymmetry of gait cycle phase between the legs, or any other known gait pathologies such as circumduction gait and hiking gait, as can be seen in FIGS. 7a and 7b. Specifically, FIG. 7a includes side, top and front view graphs of a reconstruction of circumduction gait. FIG. 7b includes side, top and front view graphs of a reconstruction of hiking gait. Both gaits share features since they exhibit events (phases) within the gait cycle. Also, these gaits include other parameters that are neither different nor absent in a normal gait, such as horizontal range parameters during various gait phases, in the circumduction gait, or vertical range parameters during various gait phases, in the hiking gait. The term “horizontal range” refers to the range in z-axis of the motion during some part of the gait cycle, since the reconstructed pattern is typically a 3d shape.

The system may treat disorders as a subtype of activity e.g. when the system computes baseline values for parameters characteristic of a given abnormal gait condition or disorder. Different parameters may also be extracted for disorders e.g. a given parameter p′ may be extracted if a system is tasked with alerting about disorder y e.g., say, circumduction, but not otherwise. For example, an end-user's inability to bend her or his knee may be detected by identifying the magnitude of the (vertical) swing; a user unable to bend his knee tends to have an abnormally high swing.

For example, walking-circumduction_gait-right_front_pants_pocket may approximately determine the motion pattern, for example, the abnormal gait condition known as circumduction, and therefore more parameters specific to a given abnormal gait condition may be extracted, such as, for circumduction, the parameter of how horizontal the swing of circumduction gait is. Abnormal gait detection allows trends of (known) pathologies (for example, during recovery from that pathology) to be tracked. Abnormal gait detection also yields detection of an (undiagnosed) pathology or abnormality that a mobile device-bearing individual is developing.

The system may treat disorder detection similarly to activity and device's bodily position detection, e.g. by using a classifier whose classes include activity/position/disorder triplets wherein, but usually, disorder=none. Pathological gait may also be defined as an activity, e.g. a limp, or circumduction, may be so striking that there is no need to separately define “walking with a limp”, “running with a limp” or “climbing stairs with a limp”. Collecting labeled data, during system development, may involve recording gait of users who are known to suffer from each disorder supported by the system.

### Operation i

For each parameter, both general (not specific to but one device bodily position, instead available for various bodily positions, such as stride duration and cadence) and activity and/or device's bodily position specific (such as maximal thigh rotation), the distribution of the user's baseline values may be estimated.

The system may receive from an external source e.g. medical expert, a healthy or normal range for some or all gait parameter or characteristic or feature, for the entire user population.

Alternatively or in addition, for each user the system computes a baseline for each gait parameter which typically comprises a distribution of the values that the parameter assumes, when computed from the gait of a given end-user. The baseline may also comprise just an average value where the average may be computed over a running window of most recent values sampled, from this end-user's gait, for that parameter or characteristic or feature.

The system may assume a normal e.g. bell-shaped distribution for certain or all gait parameters, and can, as gait parameters for a large number of end-users accumulated in system central memory, maintain an estimate, whose accuracy (if the estimate is constantly updated) consistently increases over time, of the mean and standard deviation for each parameter's bell curve. These estimates may be computed for the entire end-user population or, if end-user metadata such as age, gender etc. is available, for sub-populations (males, females, children, elderly, etc.) and hence can determine how rare is a given value, extracted for a given gait parameter from a given user's gait.

### Operation j: Compare Extracted Parameters to Baseline

When the system collects, from a given device-bearing end-user, a sequence of consistent similar values for a certain parameter that differ from a stored baseline value (e.g. probability distribution of a parameter's values, used to estimate probability of an observed parameter), for that user and that parameter (e.g. by checking if the likelihood, given the known joint probability of the collected sequence of the values, is lower than some threshold in which case the end-user's current parameter values are abnormal relative to the end-user's baseline or typical previous values for that parameter), the system may conclude that something has changed in this end-user's movement pattern and may provide a suitable alert e.g. an audible alert or a warning text message.

It is appreciated that likelihood functions are well known in the art.

According to certain embodiments, the system assumes that the parameters' values are independent given the user's baselines/distributions, and therefore the joint distribution of the parameter sequence may be obtained by multiplication of the parameters' respective probabilities.

Alternatively or in addition, the system may determine how abnormal is the data of a given end-user, relative to population norms and on that basis (and/or on the basis of the stored baseline, as described above) the system may either generate an output warning, or not, by defining a threshold which is considered to differentiate normal from abnormal.

Operation k: System Interaction with User Having Known Gait-Disorder

Interaction of the system herein with a user, e.g. to a recovering gait-disorder diagnosed user, may be initiated under different conditions, depending on the specific use-case. Two typical examples are provision of an offline progress report e and provision of real-time feedback. A progress report may be generated for the user on a regular basis, and may present the user's baseline e.g. by storing average and standard for a distribution which is assumed to be normal (bell-curve). In the general case, the system typically does not assume all parameters are normally distributed. If the system assumes that a distribution could be more complicated than the bell curve, the distribution itself may be stored in memory, rather than storing only the mean and standard deviation thereof. The distributions may then be used by the system to compute how likely a certain parameter is to have a certain value which it currently has (the probability of the parameter's just recently sampled value).

Alternatively or in addition, a progress report generated for a user may present analysis of the user's gait disorders and/or spatio-temporal parameters. Real-time feedback may be positive if a user moves as expected or may be constructive otherwise. The expected movement may be the user's baseline, or may be a desirable gait defined by a physiotherapist.

Operation k: System Action

The system may be used for different applications based on its analysis. Some example use-cases are described herein, but these are not intended to be limiting.

The system herein may be implemented as one or more end-user apps.

Interaction of the system herein with a user, e.g. to a recovering gait-disorder diagnosed user, or even a healthy user, may be initiated under different conditions, depending on the specific use-case. Two typical examples are provision of an offline progress report and provision of real-time feedback.

A progress report may be generated for the user on a regular basis. The system typically includes a smartphone/web app as an addition. In this app, the user can access a screen that shows a list of recorded motions that the system identifies as motions (as described with reference to operation c). When clicking a recorded motion, the user can see the entire analysis performed by the system and all of the features extracted (e.g. as described in operations f+g). The system also typically depicts the healthy or normal range of each gait parameter or characteristic or feature, e.g. as defined by medical experts or as measured by the system itself, over a user population or over a population of users not identified to suffer from abnormalities, and the user's baseline, i.e. average value for that gait parameter or characteristic or feature, relative to that normal range for the same parameter or characteristic or feature.

Real-time feedback may be provided by the app at any time one of the features (during a motion analyzed in real-time) deviates from its baseline value. The system adapts to the user's preferences, i.e. users that do not respond to feedback will receive less feedback and users that do respond to feedback will receive more feedback. Response to feedback means an immediate change of the motion or using the app actively to ask for more data.

Use-cases include but are not limited to the following:

1. Any gait analysis, typically continuous and seamless, using any smart device that contains a MIMU/IMU system, such as a smartphone, smartwatch or any dedicated sensor.

2. A system that uses gait monitoring to extract spatio-temporal parameters of the gait.

3. The use of a spatio-temporal reconstruction (aka preprocessing) to detect the user's activity thereby to extract spatio-temporal parameters at a higher accuracy.

4. Constructing a personalized, comprehensive analysis of the gait signature of healthy people. This may be used for all or any subset of the following:
a. Identify a user, e.g. for biometric identification purposes, or to detect when a stranger is carrying someone else's phone.
b. Build a personalized, comprehensive analysis of the gait signature of people that attend physical therapy, including gait dysfunctions.
c. Allow therapists to monitor patients' gait during day-to-day activities.
d. Provide real-time feedback on gait.
e. Analyze different physical activities such as running, martial arts, yoga, Pilates, swimming, and provide feedback for professionals and non-professionals. For example, an app could be provided to support physical activity trainers such as yoga teachers, or an end-user may initiate a record, perhaps during a specific exercise.
f. Support medical research on the connection between gait and other health-related issues e.g. by generating databases of patients' gaits and using these to find correlations in various health issues, such as but not limited to orthopedic or neurologic diseases and disorders.
g. Tele-diagnosis of mild conditions, such as people who toe-in, weak ankles.
h. Correcting posture, e.g. for teens, or any other orthopedic self-improvement app which provides an end-user with biofeedback regarding a mild orthopedic disorder he seeks to improve or overcome.
5. Analysis on the device itself rather than on the cloud, to provide privacy, real-time, or network offline capabilities.

The offline information such as medical history, age, gender collected in operation a may be used to generate a prior of risk probabilities that integrates with the decision process of operation k. For example, if an old man starts to limp, this may be deemed a higher risk situation than a limping young man. Thus, based on the user's age, the system logic may decide how fast to initiate an interaction e.g. in near-real time, as opposed to daily or weekly e.g. by email.

Sensor timing: The magneto-inertial sensor need not always be turned on e.g. in order to optimize battery usage. Operation flow may, instead, start only when new data is produced/sampled. Thus, optionally, the sensor is triggered using a timing mechanism which may depend on any or all of:
i. current activity,
ii. confidence of the gait assessment accuracy, and
iii. suspected risk.

According to certain embodiments, the cellphone logic is configured such that the cellphone's sensors are not always on, and, instead, sensors are turned on by the cellphone's operation system e.g. by various requests.

i. Activity: when the user is not active, recording his motion is useless, however, periods of motion are an opportunity for the system to learn his motions. Thus, when the user is not active, the system may be periodically triggered at a low rate, such as, say, every 15 minutes, only to indicate when he becomes active, and, once this low rate activation results in a discovery by the system that the user is moving/has become active, the rate may increase e.g. to, say, every 3 minutes, or even initiate a longer record, since a longer record can provide additional and/or more accurate information.

ii. Assessment confidence: Parameter values which describe the motion pattern can vary because of temporary arbitrary behavior, such as clumsy gait by the user due to inferior surroundings e.g. when the end-user avoids an obstacle or traverses rough ground. When parameter values vary within a single motion record, between different segments, the confidence in their values is low. Hence, if parameter values are found to vary within a single motion record, the system may initiate another record to achieve more valid results.

iii. Suspected risk: In some cases, the system may temporarily increase the rate of periodic triggers. Generally, these cases are when the system detects a suspicion of risk, for instance, an imbalance or a slight limp, which are suggestive of a risk of stroke. In that case, symptoms e.g. changes in the user's motion, manifested by the extracted parameters, will become more distinguishable progressively, and thus it is desirable for the system to track them closely, particularly in cases e.g. stroke for which rapid treatment is deemed essential.

It is appreciated that the system herein may be implemented as a software-only system which utilizes legacy hardware. For example, Android has an API via which the system herein may access data streaming from the sensors on android phones, and similarly for IOS. Software components of the system herein may be deployed on the device using any suitable technology, such as downloading onto a legacy device or being installed a priori, built-in with the device in the factory.

The system herein typically includes a user interface via which the user may be prompted to give prior consent to any potential privacy violation such as communicating stroke alerts to emergency services.

The system herein may comprise a mobile app or cell app or web app that interacts with an end-user or subscriber. The system herein may also be a web service of other software which generates patient reports for therapists or other health workers or insurance-related entities. The system herein may also comprise a software tool for performing gait and motion studies.

The embodiments herein assume raw data is collected from a wearable device's magneto-inertial sensor/s such as but not limited to accelerometers e.g. three-axial accelerometer/s and/or gyroscope/s and/or magnetometer/s.

Typically but not necessarily, the system calibrates the orientation of the mobile device's rotation to, say, east-north-sky.

Alternatively or in addition, the system may receive raw data from GPS device/s for calibrations (stride length for instance).

Alternatively or in addition, the system may receive raw data from barometer/s (e.g. for computing the mobile device's height above ground).

The terms processor or controller or module or logic as used herein are intended to include computer microprocessors which typically have digital memory and processing capacity, such as those available from, say Intel and Advanced Micro Devices (AMD), and any operation or functionality or computation or logic described herein may be implemented entirely or in any part on any suitable circuitry including any such computer microprocessor/s as well as in firmware or in hardware or any combination thereof.

It is appreciated that terminology such as “mandatory”, “required”, “need” and “must” refer to implementation choices made within the context of a particular implementation or application described herewithin for clarity and are not intended to be limiting since in an alternative implementation, the same elements might he defined as not mandatory and not required or might even be eliminated altogether.

Components described herein as software may, alternatively, he implemented wholly or partly in hardware and/or firmware, if desired, using conventional techniques, and vice-versa. Each module or component or processor may be centralized in a single physical location or physical device or distributed over several physical locations or physical devices.

Included in the scope of the present disclosure, inter alia, are electromagnetic signals in accordance with the description herein. These may carry computer-readable instructions for performing any or all of the operations of any of the methods shown and described herein, in any suitable order including simultaneous performance of suitable groups of operations as appropriate. Included in the scope of the present disclosure, inter alia, are machine-readable instructions for performing any or all of the operations of any of the methods shown and described herein, in any suitable order; program storage devices readable by machine, tangibly embodying a program of instructions executable by the machine to perform any or all of the operations of any of the methods shown and described herein, in any suitable order i.e. not necessarily as shown, including performing various operations in parallel or concurrently rather than sequentially as shown; a computer program product comprising a computer usable medium having computer readable program code, such as executable code, having embodied therein, and/or including computer readable program code for performing, any or all of the operations of any of the methods shown and described herein, in any suitable order; any technical effects brought about by any or all of the operations of any of the methods shown and described herein, when performed in any suitable order; any suitable apparatus or device or combination of such, programmed to perform, alone or in combination, any or all of the operations of any of the methods shown and described herein, in any suitable order; electronic devices each including at least one processor and/or cooperating input device and/or output device and operative to perform e.g. in software any operations shown and described herein; information storage devices or physical records, such as disks or hard drives, causing at least one computer or other device to be configured so as to carry out any or all of the operations of any of the methods shown and described herein, in any suitable order; at least one program pre-stored e.g. in memory or on an information network such as the Internet, before or after being downloaded, which embodies any or all of the operations of any of the methods shown and described herein, in any suitable order, and the method of uploading or downloading such, and a system including server/s and/or client/s for using such; at least one processor configured to perform any combination of the described operations or to execute any combination of the described modules; and hardware which performs any or all of the operations of any of the methods shown and described herein, in any suitable order, either alone or in conjunction with software. Any computer-readable or machine-readable media described herein is intended to include non-transitory computer- or machine-readable media.

Any computations or other forms of analysis described herein may be performed by a suitable computerized method. Any operation or functionality described herein may be wholly or partially computer-implemented e.g. by one or more processors. The invention shown and described herein may include (a) using a computerized method to identify a solution to any of the problems or for any of the objectives described herein, the solution optionally including at least one of a decision, an action, a product, a service or any other information described herein that impacts, in a positive manner, a problem or objectives described herein; and (b) outputting the solution.

The system may, if desired, be implemented as a Network e.g. web-based system employing software, computers, routers and telecommunications equipment as appropriate.

Any suitable deployment may be employed to provide functionalities e.g. software functionalities shown and described herein. For example, a server may store certain applications, for download to clients, which are executed at the client side, the server side serving only as a storehouse. Any or all functionalities e.g. software functionalities shown and described herein may be deployed in a cloud environment. Clients e.g. mobile communication devices such as smartphones may be operatively associated with, but external to the cloud.

The scope of the present invention is not limited to structures and functions specifically described herein and is also intended to include devices which have the capacity to yield a structure, or perform a function, described herein, such that even though users of the device may not use the capacity, they are, if they so desire, able to modify the device to obtain the structure or function.

Any “if -then” logic described herein is intended to include embodiments in which a processor is programmed to repeatedly determine whether condition x, which is sometimes true and sometimes false, is currently true or false and to perform y each time x is determined to be true, thereby to yield a processor which performs y at least once, typically on an “if and only if” basis e.g. triggered only by determinations that x is true and never by determinations that x is false.

Any determination of a state or condition described herein, and/or other data generated herein, may be harnessed for any suitable technical effect. For example, the determination may be transmitted or fed to any suitable hardware, firmware or software module, which is known or which is described herein to have capabilities to perform a technical operation responsive to the state or condition. The technical operation may for example comprise changing the state or condition or may more generally cause any outcome which is technically advantageous given the state or condition or data, and/or may prevent at least one outcome which is disadvantageous given the state or condition or data. Alternatively or in addition, an alert may be provided to an appropriate human operator or to an appropriate external system.

Features of the present invention, including operations, which are described in the context of separate embodiments, may also be provided in combination in a single embodiment.

Any teachings of features, embodiments, implementations etc. herein may be combined into a single embodiment, except where the specification specifically indicates that certain teachings are mutually contradictory and cannot be combined.

A system embodiment is intended to include a corresponding process embodiment and vice versa. Also, each system embodiment is intended to include a server-centered “view” or client centered “view”, or “view” from any other node of the system, of the entire functionality of the system, computer-readable medium, apparatus, including only those functionalities performed at that server or client or node. Features may also be combined with features known in the art and particularly although not limited to those described in the Background section or in publications mentioned therein.

Conversely, features of the invention, including operations, which are described for brevity in the context of a single embodiment or in a certain order, may be provided separately or in any suitable sub-combination, including with features known in the art (particularly although not limited to those described in the Background section or in publications mentioned therein) or in a different order. “e.g.” is used herein in the sense of a specific example which is not intended to be limiting. Each method may comprise all or any subset of the operations illustrated or described, suitably ordered e.g. as illustrated or described herein.

Devices, apparatus or systems shown coupled in any of the drawings may in fact be integrated into a single platform in certain embodiments, or may be coupled via any appropriate wired or wireless coupling such as but not limited to optical fiber, Ethernet, radio communication, Wireless LAN, HomePNA, power line communication, VR application, cell phone, Smart Phone (e.g. iPhone), Tablet, Laptop, PDA, Blackberry GPRS, Satellite including GPS, or other mobile delivery. It is appreciated that in the description and drawings shown and described herein, functionalities described or illustrated as systems and sub-units thereof can also be provided as methods and operations therewithin, and functionalities described or illustrated as methods and operations therewithin can also be provided as systems and sub-units thereof. The scale used to illustrate various elements in the drawings is merely exemplary and/or appropriate for clarity of presentation and is not intended to be limiting.

Any suitable communication may be employed between separate units herein e.g. wired data communication and/or in short-range radio communication with sensors such as cameras e.g. via WiFi, Bluetooth or Zigbee.

It is appreciated that implementation via a cellular app as described herein is but an example and instead, embodiments of the present invention may be implemented, say, as a smartphone SDK; as a hardware component; as an STK application, or as suitable combinations of any of the above.

Any processing functionality illustrated (or described herein) may be executed by any device having a processor, such as but not limited to a mobile telephone, set-top box, TV, remote desktop computer, game console, tablet, mobile e.g. laptop or other computer terminal, embedded remote unit, which may either be networked itself (may itself be a node in a conventional communication network e.g.) or may be conventionally tethered to a networked device (to a device which is a node in a conventional communication network or is tethered directly or indirectly/ultimately to such a node).

1. A gait monitoring system operative to monitor gait of an end-user bearing a wearable device equipped with at least one magneto-inertial sensor, the system comprising:
a processor configured to receive raw sensor data from the wearable device's at least one magneto-inertial sensor to extract situational data from said raw sensor data, the situational data including at least the device's bodily position relative to the end-user, to determine a gait analysis process which yields at least one parameter characterizing the end-user's gait, depending at least on said device's bodily position as extracted, and to compute, and generate an output indication of, said at least one parameter characterizing the end-user's gait, by running said gait analysis process as selected.

2. The system according to claim 1 wherein said situational data also comprises a classification of a physical activity in which the end-user is engaging while the magneto-inertial sensor is recording said raw sensor data.

3. The system according to claim 2 wherein, to extract the situational data, the processor operates a classifier which receives inputs including a stream of motion data or device acceleration data which may be derived from the magneto-inertial sensor and outputs one of plural classes for each input, and wherein at least some of said classes include an (end-user physical activity, device's bodily position) pair.

4. The system according to claim 1 wherein said wearable device comprises a networked communication device.

5. The system according to claim 1 wherein said processor includes logic which is responsive to each receipt of said raw sensor data and wherein said processor is operative to extract, select, and compute, triggered by said logic.

6. The system according to claim 5 wherein the logic, in at least some operational modes, triggers said processor to extract, select and compute, responsive to less than all instances of receipt of said raw sensor data.

7. The system according to claim 5 wherein at least one operational mode is provided whose internal logic triggers said processor to extract, selects and computes responsive to each instance of receipt of said raw sensor data.

8. The system according to claim 1 wherein said parameter characterizing the end-user's gait comprises an indication of whether the end-user's gait is characteristic of an end-user who is undergoing a stroke.

9. The system according to claim 1 wherein said parameter characterizing the end-user's gait comprises the end-user's walking pace.

10. The system according to claim 1 wherein said parameter characterizing the end-user's gait comprises the end-user's asymmetry.

11. The system according to claim 1 wherein said parameter characterizing the end-user's gait comprises the end-user's cadence.

12. The system according to claim 1 wherein said parameter characterizing the end-user's gait comprises the end-user's stride length.

13. The system according to claim 1 wherein the gait analysis process selected for a first bodily position extracts at least one parameter characterizing the end-user's gait, which is not extracted by the gait analysis process selected for a second bodily position.

14. The system according to claim 2 wherein the system stores an activity-specific baseline value for at least one parameter P characterizing the end-users gait and wherein said output indication comprises an indication of whether an end-user's gait's current value for P has strayed from the activity-specific baseline value stored specifically for the end-user and specifically for the activity in which the end-user is currently engaged.

15. The system according to claim 1 wherein the system stores a device's bodily position-specific baseline value for at least one parameter P characterizing the end-user's gait and wherein said output indication comprises an indication of whether an end-user's gait's current value for P has strayed from the device's bodily position-specific baseline value stored specifically for the end-user and specifically for the current device's bodily position as extracted.

16. The system according to claim 3 wherein the system stores an (end-user physical activity, device's bodily position) pair-specific baseline value for at least one parameter P characterizing the end-user's gait and wherein said output indication comprises an indication of whether an end-user's gait's current value for P has strayed from said baseline value stored specifically for the end-user and specifically for the (end-user physical activity, device's bodily position) pair most recently output by the classifier.

17. The system according to claim 4 wherein the wearable device comprises a cellular phone. 18, The system according to claim 1 and wherein the output indication includes at least one graph, in at least one spatial dimension, of the end-user's average stride as though the end-user were striding in place or on a treadmill.

19. The system according to claim 18 wherein the graph comprises a closed curve.

20. The system according to claim 18 wherein said at least one graph in at least one spatial dimension comprises 3 two-dimensional graphs.

21. The system according to claim 1 wherein said processor includes internal logic which is responsive to each receipt of said raw sensor data and wherein said processor is operative to extract, select and compute, triggered by said internal logic.

22. A gait analysis system including:
a processor which computationally manipulates raw sensor data describing an end-user's gait, to obtain at least one graph, in at least one spatial dimension, of the end-user's average stride as though the end-user were striding in place or on a treadmill; and
an output device which generates an output indication of said at least one graph.

23. A gait monitoring method operative to monitor gait of an end-user bearing a wearable device equipped with at least one magneto-inertial sensor, the method comprising providing a processor configured to receive raw sensor data from the wearable device's at least one magneto-inertial sensor to extract situational data from said raw sensor data, the situational data including at least the device's bodily position relative to the end-user, to determine gait analysis functionality which yields at least one parameter characterizing the end-user's gait, depending at least on said device's bodily position as extracted, and to compute, and generate an output indication of, said at least one parameter characterizing the end-user's gait, by running said gait analysis process as selected.

24. A computer program product, comprising a non-transitory tangible computer readable medium having computer readable program code embodied therein, said computer readable program code adapted to be executed to implement any method shown and described herein.
